(19) United States
(12) Patent Application Publication
(10) Pub. No.: US 2026/0117271 A1
(43) Pub. Date: Apr. 30, 2026

(54) SPECTRAL MONITORING OF IN VITRO TRANSCRIPTION

(71) Applicant: SANOFI PASTEUR INC., Swiftwater, PA (US)

(72) Inventors: Didier CLENET, Paris (FR); Supanee POTISOPON, Paris (FR)

(21) Appl. No.: 19/118,604

(22) PCT Filed: Oct. 9, 2023
(86) PCT No.: PCT/EP2023/077921

§ 371 Date: Apr. 4, 2025

(30) Foreign Application Priority Data
Oct. 7, 2022 (EP) 22315234.9

(51) Int. Cl. C12P 19/34; G01N 21/35; G01N 21/65

(52) U.S. Cl. CPC C12P 19/34; G01N 21/35; G01N 21/65

(57) ABSTRACT

The present invention provides a method for monitoring an in vitro transcription (IVT) reaction for the production of RNA within a reaction vessel. The method comprises obtaining a spectrum of a reactant or product during the IVT reaction and comparing the obtained spectrum to a pre-determined reference spectrum of the reactant or product of the IVT reaction. The method can be used to determine changes in the amount of the reactant or product during the IVT reaction.

### FIELD OF THE INVENTION

The present invention relates to a method of monitoring in vitro transcription (IVT) for the production of RNA (e.g., mRNA) by obtaining a spectrum of a reactant or product during the IVT reaction. The spectrum can then be compared to a pre-determined reference spectrum of the reactant or product of the IVT reaction. A change in the amount of the reactant or product is signified by a difference between the obtained spectrum and a pre-determined reference spectrum.

### BACKGROUND OF THE INVENTION

RNA-based therapeutics, including messenger RNA (mRNA)-based vaccines, have emerged as new therapeutic and prophylactic modalities that can be developed quickly over a short period of time. However, the development chain of such products currently lacks a Quality by Design (QbD) framework for their manufacturing. The major and first step in the manufacturing process is an in vitro transcription (IVT) reaction during which the RNA is transcribed from a DNA template. The template contains the sequence needed for the transcription of a specific RNA and is operationally linked to an RNA polymerase promoter. In the presence of ribonucleotides (NTPs) and an RNA polymerase, the DNA sequence is transcribed into RNA.

The lack of data during the manufacturing process necessitates the implementation of Process Analytical Technology (PAT) in order to define the Critical Quality Attributes (CQAs). The most relevant CQA to optimize during the manufacturing process is the RNA yield. Many parameters can affect the final yield of an IVT reaction. These include the concentrations of the reactants, such as the NTPs, the RNA polymerase, cofactors (e.g., Mg²⁺), and the DNA template. RNA yield can be improved by monitoring these critical process parameters (CPPs). Typically, this involves performing a large number of experiments to optimize the concentrations of individual reactants for a particular template sequence. Therefore, a need exists to reduce the number of experiments required to optimize the CPPs for RNA yield during IVT.

Moreover, current methods for monitoring an IVT reaction typically require removing an aliquot of the reaction mixture from the reaction vessel and processing it before the RNA yield can be determined. Commonly, such methods are limited to determining the amount of a single product or reactant of the IVT reaction (e.g., the amount of RNA). In addition, the presence of enzyme components can interfere with an accurate determination of the amount of RNA in the reaction vessel.

Thus, there is a need for a method that can monitor the amount of a reactant or product of an IVT reaction within a reaction vessel, without the necessity of obtaining an aliquot or removing enzyme components. Ideally, such a method can monitor multiple reactants and products of the IVT reaction simultaneously and can be used both in process optimisation as well as process control during the large-scale production of RNA.

### SUMMARY OF THE INVENTION

The present invention notably relates to a method for monitoring an in vitro transcription (IVT) reaction for the production of RNA, in particular messenger RNA (mRNA), using a spectroscopic probe within a reaction vessel.

When an incident light (e.g., a laser) irradiates a molecule, it is scattered inelastically causing a change in the initial wavelength. The inventors found that the resulting shift between the frequencies of the incident and of the scattered light can provide a molecular fingerprint of one or more reactants and/or products as an IVT reaction takes place in the reaction vessel. Spectra obtained during the reaction can automatically be transformed as concentrations of the main products and reactants (namely the RNA, NTPs, and H₂PO₄⁻ molar concentrations). This in-line method can augment the throughput of experiments during process development and can provide valuable process and quality data during the large-scale manufacturing of RNA-based therapeutics (in particular mRNA-based therapeutics, including mRNA-based vaccines). These data can be used to optimize reaction conditions or to determine whether a batch of RNA can proceed to downstream manufacturing steps, thereby reducing the time and cost associated with the large-scale manufacturing of an RNA (in particular mRNA).

In particular, the invention relates to a method for monitoring an in vitro transcription (IVT) reaction for the production of RNA, e.g., mRNA within a reaction vessel, said method comprising (i) obtaining a spectrum of a reactant or product during the IVT reaction; and (ii) comparing the spectrum obtained in step (i) to a pre-determined reference spectrum of the reactant or product of the IVT reaction, wherein a difference between the spectrum obtained in step (i) and the pre-determined reference spectrum indicates a change in the amount of the reactant or product.

In some embodiments, the reactant or product is selected from RNA, pyrophosphate (PPi), H⁺, inorganic phosphate (Pi), and a ribonucleotide (NTP). In some embodiments, the product is RNA or Pi. In some embodiments, the reactant is one or more ribonucleotides (NTPs).

In some embodiments, the method determines the change in the amount of more than one reactant or more than one product of the IVT reaction. In some embodiments, the method determines the change in the amount of more than one reactant and more than one product of the IVT reaction. In some embodiments, the more than one reactants are adenosine triphosphate (ATP) and guanosine triphosphate (GTP). In some embodiments, the more than one reactants are cytidine triphosphate (CTP) and uridine triphosphate (UTP). In some embodiments, the more than one products are RNA and Pi.

In some embodiments, the reaction vessel is a bioreactor. In some embodiments, the reaction vessel has an access port or a by-pass for insertion of a spectroscopic probe. In some embodiments, the spectroscopic probe is immersed in the solution in which the IVT reaction occurs. In some embodiments, the spectroscopic probe is not immersed in the solution in which the IVT reaction occurs. In some embodiments, the spectroscopic probe is separated from the solution by a barrier that does not interfere with detection.

In some embodiments, step (i) comprises obtaining a series of spectra over the course of the IVT reaction. In some embodiments, each spectrum in the series is obtained over a time period of 10-60 seconds. In some embodiments, the series of spectra comprises a set of at least 3, at least 5, or at least 9 spectra. In some embodiments, multiple series of spectra are obtained over the course of the IVT reaction.

In some embodiments, each of the spectra in the series is pre-processed prior to step (ii). In some embodiments, the spectra in the series are acquired successively and pre-processing comprises smoothing the spectra by applying a digital filter that fits the successively acquired spectra with a low-degree polynomial by a method of linear least squares.

In some embodiments, the spectrum or series of spectra obtained in step (i) is/are normalised relative to a wavelength region with reduced or no background noise prior to step (ii). In some embodiments, the series of spectra are normalised relative to a wavelength region with reduced or no background noise prior to any pre-processing step.

In some embodiments, step (ii) comprises a qualitative spectral comparison between the series of spectra obtained in step (i) and the pre-determined reference spectrum. In some embodiments, the qualitative spectral comparison comprises calculating a weighted spectral difference (WSD) value. In some embodiments, a WSD value of one standard deviation or less indicates that there is no significant difference between the spectrum obtained in step (i) and the pre-determined reference spectrum. In some embodiments, a WSD value of more than one standard deviation indicates that there is a significant difference between a spectrum obtained in step (i) and the pre-determined reference spectrum.

In some embodiments, the pre-determined reference spectrum correlates to a specified concentration of the reactant or product. In some embodiments, step (ii) further comprises determining the concentration of the reactant or product. In some embodiments, determining the concentration of the reactant or product comprises linear regression analysis. In some embodiments, a partial least square (PLS) model is used to determine the concentration of the reactant or product from a series of pre-determined reference spectra, wherein each spectrum of the series correlates to different concentrations of the reactant or product.

In some embodiments, the spectrum, series of spectra, and the pre-determined reference spectrum/series of pre-determined reference spectra, as applicable, are obtained using a spectrometer for vibrational spectroscopy. In some embodiments, the spectrometer is selected from a Raman spectrometer, an infrared (IR) spectrometer and a turbidimeter. In specific embodiments, the spectrometer is a Raman spectrometer.

In some embodiments, the spectrum obtained in step (i) spans a wavelength region suitable for monitoring the global evolution of multiple reactants and products during the IVT reaction. In some embodiments, the multiple reactants and products include RNA, inorganic phosphate (Pi) and one or more ribonucleotides (NTPs). In some embodiments, the wavelength region includes 300 cm⁻¹ to 3000 cm⁻¹ and the spectrum is obtained using Raman spectrometer.

In some embodiments, the spectrum obtained in step (i) spans a wavelength region specific to one product or reactant of the IVT reaction.

In some embodiments, the product is RNA. In some embodiments, the wavelength region includes 801 cm⁻¹ to 831 cm⁻¹ and the spectrum is obtained using a Raman spectrometer.

In some embodiments, the product is Pi. In some embodiments, the wavelength region includes 875 cm⁻¹ to 900 cm⁻¹ and the spectrum is obtained using a Raman spectrometer.

In some embodiments, the reactant is one or more ribonucleotides (NTPs). In some embodiments, the wavelength region comprises 600 cm⁻¹ to 1300 cm⁻¹ and the spectrum is obtained using a Raman spectrometer. In some embodiments, the wavelength region comprises or consists of 1107 cm⁻¹ to 1146 cm⁻¹ or 1113 cm⁻¹ to 1115 cm⁻¹ to determine the amount of NTPs. In some embodiments, the wavelength region comprises or consists of 633 cm⁻¹ to determine the amount of ATP. In some embodiments, the wavelength region comprises or consists of 1300 cm⁻¹ to 1600 cm⁻¹ to determine the amount of ATP and GTP. In some embodiments, the wavelength region comprises or consists of 780 cm⁻¹ to determine the amount of CTP. In some embodiments, the wavelength region comprises or consists of 786 cm⁻¹ to 789 cm⁻¹ to determine the amount of UTP. In some embodiments, the wavelength region comprises or consists of 1230 cm⁻¹ to 1245 cm⁻¹ to determine the amount of CTP and UTP.

In some embodiments, the spectrum obtained in step (i) detects the turbidity of the solution in which the IVT reaction occurs. In some embodiments, the turbidity indicates the accumulation of an insoluble precipitate. In some embodiments, the insoluble precipitate is Mg₂PPi. In some embodiments, turbidity is measured using a turbidimeter, a UV spectrometer or a nephelometer. In some embodiments, the turbidity is measured at a wavelength in the region comprising 290 nm to 410 nm (e.g., 300 nm to 350 nm), e.g., at 310 nm, 320 nm, 330 nm, 340 nm, or 350 nm using a UV spectrometer.

In some aspects, the invention also relates to a method of manufacturing an RNA, e.g., mRNA, using an in vitro transcription (IVT) reaction, said method comprising: (a) providing a DNA template comprising the nucleotide sequence of the RNA operationally linked to an RNA polymerase promoter; (b) adding the DNA template to a reaction vessel comprising an RNA polymerase and necessary reactants to initiate the IVT reaction; and (c) monitoring one or more of the reactants or products of the IVT reaction using the method of the invention as set out in the preceding paragraphs.

In some embodiments, step (c) comprises obtaining a series of spectra, wherein the spectra span a wavelength region suitable for monitoring the DNA template and/or one or more enzymatic components such as the RNA polymerase during the IVT reaction (or after its completion).

In some embodiments, the spectra obtained in step (c) are used to monitor the amount of DNA template. In some embodiments, the wavelength region obtained in step (c) comprises or consists of 500 cm⁻¹ to 710 cm⁻¹, 1325 cm⁻¹ to 1365 cm⁻¹, and/or 1585 cm⁻¹ to 1725 cm⁻¹ to determine the amount of plasmid DNA.

In some embodiments, the spectra obtained in step (c) are used to monitor the amount of RNA polymerase. In some embodiments, the RNA polymerase is SP6 RNA polymerase. In some embodiments, the wavelength region obtained in step (c) comprises or consists of 780 cm⁻¹ to 1200 cm⁻¹ and/or 1430 cm⁻¹ to 1510 cm⁻¹ to determine the amount or addition of the SP6 RNA polymerase.

In some embodiments, the method comprises adding a nuclease (e.g., DNase I) to terminate the IVT reaction. In some embodiments, the spectra obtained in step (c) are used to monitor the amount or addition of the nuclease. In some embodiments, the wavelength region obtained in step (c) comprises or consists of 450 cm⁻¹ to 520 cm⁻¹, 1000 cm⁻¹ to 1090 cm⁻¹, and/or 2915 cm⁻¹ to 3000 cm⁻¹ to determine the amount or addition of the nuclease.

In some embodiments, the method comprises adding a protease (e.g., proteinase K) to terminate the IVT reaction (e.g., by digesting the RNA polymerase), or to inactivate the nuclease. In some embodiments, the spectra obtained in step (c) are used to monitor the amount or addition of the protease. In some embodiments, the wavelength region obtained in step (c) comprises or consists of 505 cm⁻¹ to 610 cm⁻¹ (e.g., 550 cm⁻¹ to 600 cm 1), 715 cm⁻¹ to 775 cm⁻¹, and/or to 1385 cm⁻¹ to 1395 cm⁻¹ to determine the amount or addition of the protease.

In some embodiments, the one or more reactants or products is RNA. In some embodiments, the monitoring in step (i) of a method of the invention comprises obtaining a series of spectra at one or more specified time points during the IVT reaction and step (ii) comprises determining the amount, or change in the amount, of RNA between the one or more specified time points by comparing the series of spectra and the pre-determined reference spectrum.

In some embodiments, the IVT reaction is terminated if the RNA does not reach a target amount at the one or more specified time points. In some embodiments, the batch of RNA resulting from the IVT reaction is discarded.

In some embodiments, the IVT reaction is terminated if the change in the amount of RNA is less than a pre-determined value at two or more specified time points. In some embodiments, the batch of RNA resulting from the IVT reaction is discarded.

In some embodiments, the two or more specified time points are at equally-spaced intervals throughout the IVT reaction. In some embodiments, each interval is 10 minutes or less, 5 minutes or less, 1 minute or less, or 30 seconds or less.

In some embodiments, the IVT reaction is terminated if the change in the amount of RNA is about zero between at least two or more time points. In some embodiments, the at least two or more time points are at least 5 minutes apart.

In some embodiments, the IVT reaction is terminated if the spectrum of the IVT reaction obtained in step (i) diverges by more than one standard deviation from (A) the pre-determined reference spectrum of the reactant or product, or (B) a kinetic model previously determined for an IVT reaction using similar or identical conditions and reactants.

In some embodiments, the batch of RNA resulting from the IVT reaction is discarded if the spectrum of the IVT reaction obtained in step (i) diverges by more than one standard deviation from (A) the pre-determined reference spectrum of the reactant or product, or (B) a kinetic model previously determined for an IVT reaction using similar or identical conditions and reactants.

In some aspects, the invention also relates to a method of manufacturing an RNA, e.g., mRNA, using an in vitro transcription (IVT) reaction, said method comprising (a) monitoring the production of the RNA within a reaction vessel by (i) obtaining a spectrum of the RNA during the IVT reaction to determine a first value; and (ii) comparing the first value obtained in step (i) to a second value derived from a pre-determined reference spectrum of RNA; and (b) purifying the RNA if the first value is equal to, or exceeds, the second value.

In some embodiments, the second value corresponds to a target concentration. In some embodiments, the target concentration is at least 3 g/L.

In some embodiments, steps (i) and (ii) are repeated at equally spaced intervals and the RNA is purified if the first value obtained at each interval is equal to or exceeds the second value at corresponding intervals derived from the pre-determined reference spectrum.

In some embodiments, the first and second values are considered equal if they are within one standard deviation of each other.

In some embodiments, the reaction vessel is a bioreactor. In some embodiments, the reaction vessel has an access port or a by-pass for insertion of a spectroscopic probe. In some embodiments, the spectroscopic probe is immersed in the solution in which the IVT reaction occurs. In some embodiments, the spectroscopic probe is not immersed in the solution in which the IVT reaction occurs. In some embodiments, the spectroscopic probe is separated from the solution by a barrier that does not interfere with detection.

In some embodiments, the reaction vessel has an access port for the addition of reactants during the IVT reaction. In some embodiments, the reactants in the IVT reaction include magnesium (Mg²⁺) and NTPs. In some embodiments, the IVT reaction is supplemented at least once with NTPs during the course of manufacturing the RNA. In some embodiments, the IVT reaction is supplemented periodically with NTPs during the course of manufacturing the RNA.

In some embodiments, the IVT reaction is supplemented with NTPs when the concentration of NTPs approaches depletion. In some embodiments, depletion is approached when the NTPs are at a concentration of no more than 5% of the concentration of NTPs that was present when the IVT reaction was initiated. In some embodiments, depletion is approached when an NTP is at a concentration of no more than 5 mM. In some embodiments, depletion is approached when an NTP is at a concentration of no more than 3 mM.

In some embodiments, the IVT reaction is supplemented continuously with NTPs during the course of manufacturing the RNA.

In some embodiments, each NTP is present at a concentration of 1-10 mM, 1-6 mM, 2-6 mM, or 3-6 mM when the IVT reaction is initiated. In some embodiments, the IVT reaction is supplemented with NTPs to maintain or restore the concentration at/to the concentration of NTPs that was present when the IVT reaction was initiated. In some embodiments, the IVT reaction is supplemented with NTPs to maintain the concentration of each NTP within a range of 20%-100%, 20%-75%, or 25%-50% that was present when the IVT reaction was initiated.

In some embodiments, the total NTP concentration in the IVT reaction is maintained above a lower limit of 2 mM. In some embodiments, the total NTP concentration in the IVT reaction is maintained at 10 mM to 20 mM.

In some aspects, the invention also provides a method of manufacturing an RNA using an in vitro transcription (IVT) reaction, said method comprising (a) providing a DNA template comprising the nucleotide sequence of the RNA operationally linked to an RNA polymerase promoter; (b) adding the DNA template to a reaction vessel comprising an RNA polymerase and necessary reactants to initiate the IVT reaction; and (c) monitoring the IVT reaction by obtaining a series of spectra, wherein the spectra span a wavelength region suitable for monitoring the DNA template and/or the RNA polymerase during the IVT reaction.

In some embodiments, the spectra obtained in step (c) are used to monitor the amount of DNA template. In some embodiments, the wavelength region obtained in step (c) comprises or consists of 500 cm⁻¹ to 710 cm⁻¹, 1325 cm⁻¹ to 1365 cm⁻¹, and/or 1585 cm⁻¹ to 1725 cm⁻¹ to determine the amount of plasmid DNA.

In some embodiments, the spectra obtained in step (c) are used to monitor the amount of RNA polymerase. In some embodiments, the RNA polymerase is SP6 RNA polymerase. In some embodiments, the wavelength region obtained in step (c) comprises or consists of 780 cm⁻¹ to 1200 cm⁻¹ and/or 1430 cm⁻¹ to 1510 cm⁻¹ to determine the amount or addition of the SP6 RNA polymerase.

In some embodiments, the method comprises adding a nuclease (e.g., DNase I) to terminate the IVT reaction. In some embodiments, the spectra obtained in step (c) are used to monitor the amount or addition of the nuclease. In some embodiments, the wavelength region obtained in step (c) comprises or consists of 450 cm⁻¹ to 520 cm⁻¹, 1000 cm⁻¹ to 1090 cm⁻¹, and/or 2915 cm⁻¹ to 3000 cm⁻¹ to determine the amount or addition of the nuclease.

In some embodiments, the method comprises adding a protease (e.g., proteinase K) to terminate the IVT reaction or nuclease activity. In some embodiments, the spectra obtained in step (c) are used to monitor the amount or addition of the protease. In some embodiments, the wavelength region obtained in step (c) comprises or consists of 505 cm⁻¹ to 610 cm⁻¹, (e.g., 550 cm⁻¹ to 600 cm⁻¹), 715 cm⁻¹ to 775 cm⁻¹, and/or to 1385 cm⁻¹ to 1395 cm⁻¹ to determine the amount or addition of the protease.

### BRIEF DESCRIPTION OF THE DRAWINGS

Embodiments of the invention will be described, by way of example, with reference to the following drawings, in which:

FIG. 1 is an illustration of the spectral attributions of Raman spectrometry profiles during an IVT reaction over a selected wavelength region of 800-1300 cm⁻¹. The dotted areas framed by dashed lines underneath the spectra represent wavelength regions in which a change in the amount of RNA, NTPs (including CTP and UTP), or the reaction by-product H₂PO₄⁻ or Pi can be detected, as shown in the figure. The peaks are labelled to indicate representative wavelengths for the detection of these reactants and products by Raman spectroscopy. The dashed line represents the first recorded spectrum after 8 minutes from the beginning of the IVT reaction. The dark-grey solid line is the last spectrum recorded of the IVT reaction after 90 minutes. The light-grey lines represent intermediate spectra recorded during the IVT reaction.

FIG. 2 illustrates the evolution of weighted spectral difference (WSD) values calculated from series of spectra obtained with a Raman spectrometer for (a) the 801-831 cm⁻¹ wavelength region (RNA) and (b) the 1107-1146 cm⁻¹ wavelength region (NTPs). The spectra were obtained during IVT reactions performed at 37° C. The results, obtained at a 2 mL scale, are shown as a solid line (average of n=3)± one standard deviation (shown as dashed lines). The results from an illustrative experiment performed at a 250 mL scale (Ambr®250, n=1) are shown as a solid line with open circles.

FIG. 3 shows RNA concentrations during an IVT reaction determined either in-line by a univariate PLS model based on Raman spectrometry data obtained from within a reaction vessel (dotted line) or off-line by RiboGreen assay from aliquots taken from the reaction vessel (dashed line).

FIG. 4 shows real-time concentrations of purine-based NTPs (ATP-GTP), pyrimidine-based NTPs (CTP-UTP), and inorganic phosphate (Pi) determined by univariate PLS models based on a series of Raman spectra obtained during a representative IVT reaction.

FIG. 5 illustrates concentration profiles of the reactants (ATP-GTP, CTP-UTP) and products (RNA, Pi, and PPi) of an IVT reaction based on a series of Raman spectra obtained during a representative IVT reaction (closed circles), overlaid with the predictions of a kinetic model (solid lines). RNA concentrations were determined both by Raman spectrometry (open circles) and by RiboGreen assay (open triangles).

FIG. 6 illustrates predictions of progress of turbidity during IVT reactions performed at 31° C., 37° C., and 42° C. In panel (a), turbidity predictions are displayed as lines for up to 5 hours at 31° C., 37° C., and 42° C., with predictive bands representing 95% prediction interval shown as dashed lines. Turbidity data (absorbance: 320 nm) used for kinetic modelling are displayed as filled circles. Additional experimental data obtained after 1.5 hours are displayed as open circles, and were not used for kinetic modelling. Panel (b) shows a time-temperature-transformation (TTT) diagram covering a 1%-99% isoconversion range. The area filled in grey shows a time-temperature domain preventing significant turbidity during an IVT reaction.

FIG. 7 illustrates RNA concentrations obtained during IVT reactions at three different batch sizes. Specifically, IVT reactions produced 150 mg, 1 g, or 20 g of mRNA, as represented by open circles, closed squares, and open triangles, respectively. Concentrations were determined by a univariate PLS model based on Raman spectrometry as described in Example 3. The IVT reactions were monitored for a period of 2 hours, as indicated in the figure.

FIG. 8 illustrates the concentrations of the products RNA and PPi and two of the nucleotide reactants (GTP and ATP) during an IVT reaction performed at different batch sizes. Panels (a)-(c) of FIG. 8 show the concentrations of RNA (mg/mL), PPi (mM) and GTP-ATP (mM), respectively, as determined using a univariate PLS model based on Raman spectrometry data (see Example 3). The reactions were monitored for a period of approximately 4 hours, as indicated in the figures. IVT reactions were performed at an mRNA batch size of 1 g or 20 g, as represented by open circles or closed squares, respectively. Numbered lines indicate the reaction stage. 1 is the IVT reaction stage, 2 is the termination stage (initiated by the addition of DNase I and followed by the addition of proteinase K), and 3 is the quenching stage (initiated by the addition of DTT).

FIG. 9 illustrates RNA concentrations obtained during an IVT reaction producing 1 g of mRNA measured in-line by Raman spectrometry or off-line by the RiboGreen assay (depicted by filled circles or open squares, respectively). Numbered lines indicate the reaction stage. 1 is the IVT reaction stage, 2 is the termination stage (initiated by the addition of DNase I and followed by the addition of proteinase K), and 3 is the quenching stage (initiated by the addition of DTT). The reaction was monitored for a period of approximately 4 hours, as indicated in the figure.

FIG. 10 is an illustration of the spectral attributions of ATP and GTP obtained from a Kaiser Raman spectrometer. In panels (a) and (b) of FIG. 10, four different concentrations of ATP and GTP were tested in a spike experiment to identify the wavelength regions in which ATP and GTP are identifiable. Panels (a) and (b) represent spectra obtained for a wavelength region of 1550 cm⁻¹ to 1600 cm⁻¹ and 1550 cm⁻¹ to about 1605 cm⁻¹, respectively. In panels (c) and (d) of FIG. 10, regions where ATP and GTP were identified were overlaid. Panel (c) represents spectra obtained for a wavelength region of 1550 cm⁻¹ to 1590 cm⁻¹, and panel (d) represents spectra obtained for a wavelength region of about 640 cm⁻¹ to about 775 cm⁻¹, respectively.

FIG. 11 is an illustration of the spectral attributions of plasmid DNA as determined by Raman spectrometry. As shown in panel (a) of FIG. 11, IVT reaction mixtures comprising spiked plasmid DNA at concentrations of 0 mg/mL, 0.075 mg/mL, 0.15 mg/mL, 0.3 mg/mL, and 0.6 mg/mL, respectively, were monitored across the wavelength region of 400 cm⁻¹ to 3000 cm⁻¹. Regions A-C are framed and shown separately in panels (b)-(d) of FIG. 11. Panel (b) represents spectra obtained for the wavelength region of 505 cm⁻¹ to 705 cm⁻¹. Panel (c) represents spectra obtained for the wavelength region of 1325 cm⁻¹ to 1370 cm⁻¹. Panel (d) represents spectra obtained for the wavelength region of 1585 cm⁻¹ to 1720 cm⁻¹.

FIG. 12 is an illustration of the spectral attributions of DNase I as determined by Raman spectrometry. As shown in panel (a) of FIG. 12, IVT reaction mixtures comprising spiked DNase I at concentrations of 0 ku/mL, 0.063 ku/mL, 0.125 ku/mL, 0.25 ku/mL, and 0.5 ku/mL, respectively, were monitored across the wavelength region of 400 cm⁻¹ to 3000 cm⁻¹. Regions A-C are framed and shown separately in panels (b)-(d) of FIG. 12. Panel (b) represents spectra obtained for the wavelength region of 440 cm⁻¹ to 515 cm⁻¹. Panel (c) represents spectra obtained for the wavelength region of about 1005 cm⁻¹ to about 1085 cm⁻¹. Panel (d) represents spectra obtained for the wavelength region of 2915 cm⁻¹ to 3000 cm⁻¹.

FIG. 13 is an illustration of the spectral attributions of SP6 RNA polymerase as determined by Raman spectrometry. As shown in panel (a) of FIG. 11, IVT reaction mixtures comprising spiked SP6 RNA polymerase concentrations of 0 mg/mL, 0.045 mg/mL, 0.09 mg/mL, 0.18 mg/mL, and 0.36 mg/mL, respectively, were monitored across the wavelength region of 350 cm⁻¹ to 1700 cm⁻¹. Regions A and B are framed and shown separately in panels (b) and (c) of FIG. 13. Panel (b) represents spectra obtained for the wavelength region of 780 cm⁻¹ to 1140 cm⁻¹. Panel (c) represents spectra obtained for the wavelength region of 1430 cm⁻¹ to 1500 cm⁻¹.

### DEFINITIONS

In order for the present invention to be more readily understood, certain terms are first defined below. Additional definitions for the following terms and other terms are set forth throughout the specification.

As used in this specification and the appended claims, the singular forms “a”, “an”, and “the” include plural referents and plural terms include the singular, unless the context clearly dictates otherwise. For example, “a spectrum” is understood to represent one or more spectra. As such, the terms “a” (or “an”), “one or more”, and “at least one” can be used interchangeably herein.

Unless specifically stated or obvious from context, as used herein, the term “or” is understood to be inclusive and covers both “or” and “and”. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising”, otherwise analogous aspects described in terms of “consisting of” and/or “consisting essentially of” are also provided.

As used herein, the term “about” refers to an interval of accuracy that a person skilled in the art will understand to still ensure the technical effect of the feature in question. The term indicates a deviation from the indicated numerical value of +10%. In some embodiments, the deviation is +5% of the indicated numerical value. In certain embodiments, the deviation is +1% of the indicated numerical value.

The term “reaction vessel” refers to any container suitable for performing an IVT reaction. The reaction vessel contains the reactants, enzyme components and any additional components (such as buffering reagents, etc.) required to perform the IVT reaction. The volume of the reaction vessel and/or its configuration may depend on the scale of the IVT reaction and/or the application for which the RNA is prepared. Suitable reaction vessels may be made of glass, plastic, or stainless steel. In some embodiments, the reaction vessel can be sterilised and sealed to avoid contamination (e.g., a single-use sterilisable and sealable plastic bag).

As used herein, the term “bioreactor” refers to a reaction vessel that may include a means of heating and/or providing agitation to the reaction mixture, or is adapted to be operably linked to such means. Often bioreactors also include ports for the addition of reactants and/or for the insertion of a probe. In some embodiments, the bioreactor may include a by-pass for the insertion of spectroscopic probe.

As used herein the term “RNA” refers to any polyribonucleotide. More typically, in the context of the present invention, the term refers to a polyribonucleotide with a length of at least 100 ribonucleotides, e.g., at least 200 ribonucleotides or at least 400 ribonucleotides. In addition to messenger RNA (mRNA), this may include ribosomal RNAs, ribozymes, riboswitches, and/or other long non-coding RNAs (lncRNAs), e.g., Kcnq1ot1, Xlsirt, Xist, and HOTAIR.

As used herein, the term “mRNA” refers to a polyribonucleotide that encodes at least one polypeptide. mRNA may contain one or more coding and non-coding regions (e.g., a 5′ untranslated region and a 3′ untranslated region). mRNA as used herein encompasses both modified and unmodified RNA. For example, mRNA can comprise one or more nucleoside analogues, such as analogues having chemically modified bases or sugars, backbone modifications, etc. An mRNA sequence is presented in the 5′ to 3′ direction unless otherwise indicated. A typical mRNA comprises a 5′ cap, a 5′ untranslated region (5′ UTR), a protein-coding region, a 3′ untranslated region (3′ UTR), and a 3′ tail. In some embodiments, the tail structure is a poly(C) tail. More typically, the tail structure is a poly(A) tail

As used herein the term “sequence-optimized” is used to describe a nucleotide sequence that is modified relative to a naturally-occurring or wild-type nucleic acid. Such modifications may include, e.g., codon optimization and/or the use of 5′ UTRs and 3′ UTRs which are not normally associated with the naturally-occurring or wild-type nucleic acid. As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by removing with filters less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

As used herein, the term “spectrum” refers to the plurality of detected signals as light emission or absorption from a sample which are recorded with a spectrometer either across one wavelength at more than one time point or across two or more wavelengths at one or more time points. For example, a spectrum may be obtained at a single wavelength over a period of time, e.g., for the duration of the IVT reaction. In some embodiments, more than one spectrum is obtained at more than one wavelength. In a typical embodiment, more than one spectrum is obtained at more than one wavelength at more than one time point during the IVT reaction.

As used herein, the term “template DNA” (or “DNA template”) relates to a DNA molecule comprising a nucleotide sequence encoding an RNA transcript to be synthesized by in vitro transcription (IVT). The template DNA is used as template for IVT in order to produce the RNA transcript encoded by the template DNA. The template DNA comprises all elements necessary for IVT, particularly a promoter element for binding of a DNA-dependent RNA polymerase, such as, e.g., T3, T7, or SP6 RNA polymerase, which is operably linked to the DNA sequence encoding a desired RNA transcript. Furthermore the template DNA may comprise primer binding sites 5′ and/or 3′ of the DNA sequence encoding the RNA transcript to determine the identity of the DNA sequence encoding the RNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleotide sequence encoding the desired RNA transcript.

Unless otherwise defined herein, technical and scientific terms used herein have the same meaning as commonly understood by one of ordinary skill in the art to which this application belongs and as commonly used in the art to which this application belongs. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Throughout this specification and embodiments, the words “have” and “comprise”, or variations such as “has”, “having”, “comprises”, or “comprising”, will be understood to imply the inclusion of a stated integer or group of integers but not the exclusion of any other integer or group of integers.

All publications and other reference materials referenced herein are hereby incorporated by reference in their entirety. Although a number of documents are cited herein, this citation does not constitute an admission that any of these documents forms part of the common general knowledge in the art.

### DETAILED DESCRIPTION OF THE INVENTION

The invention relates to a method for monitoring an in vitro transcription (IVT) reaction for the production of RNA, in particular messenger RNA (mRNA), within a reaction vessel, said method comprising (i) obtaining a spectrum of a reactant or product during the IVT reaction; and (ii) comparing the spectrum obtained in step (i) to a pre-determined reference spectrum of the reactant or product of the IVT reaction. A difference between the spectrum obtained in step (i) and the pre-determined reference spectrum indicates a change in the amount of the reactant or product.

The method of the invention is particularly advantageous because the amount of a reactant or product can be monitored within the reaction vessel. Unlike some prior art methods, the method of the invention does not require taking an aliquot or removing enzyme components from the IVT reaction prior to obtaining a spectrum. Moreover, information about the amount of a reactant or product in the IVT reaction can be provided very rapidly. In some embodiments, the amount of RNA in the reaction vessel can be monitored essentially in real time. Another advantage of the invention is that it allows the monitoring of multiple reactants and/or products simultaneously. In some embodiments, the spectrum obtained in step (i) provides information about the amount of one or more reactants (e.g., NTPs) as well as the main product (RNA) and by-products of the IVT reaction (e.g., PPi, Pi, and/or H′).

### In Vitro Transcription

“In vitro transcription” or “IVT” refers to the process whereby transcription occurs in vitro (i.e., in an artificial environment such as a reaction vessel rather than within an organism) to produce a synthetic RNA product. An IVT reaction is typically performed in the presence of an RNA polymerase and a template. The template is typically a DNA template, e.g., a linearized or circular plasmid. In some embodiments, the template (e.g., the DNA template) is monitored during the IVT reaction. In some embodiments, the amount of the template (e.g., the DNA template) is monitored in the reaction vessel by acquiring a spectrum from which information about the amount of the template can be obtained.

The DNA template comprises a nucleotide sequence that is operationally linked to an RNA polymerase promoter. In some embodiments, the promoter is an SP6 RNA polymerase promoter. In other embodiments, the promoter is a T7 RNA polymerase promoter. In other embodiments, the promoter is a T3 RNA polymerase promoter. In some embodiments, the RNA polymerase, e.g., SP6, T7, or T3 RNA polymerase, is monitored during the IVT reaction. In some embodiments, the amount of the RNA polymerase is monitored in the reaction vessel by acquiring a spectrum from which information about the amount of the template can be obtained.

In some embodiments, the DNA template may be optimized to facilitate more efficient transcription and/or downstream translation. For example, the DNA template may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA template may be optimized regarding cryptic splice sites, secondary structure of the RNA transcript, stable free energy of the RNA transcript, repetitive sequences, instability motifs, and/or other elements relevant to RNA processing and stability; the DNA template may be optimized regarding codon usage bias, codon adaptability, internal chi sites, ribosomal binding sites (e.g., IRES), premature poly(A) sites, Shine-Dalgarno (SD) sequences, and/or other elements relevant to translation; and/or the DNA template may be optimized regarding codon context, codon-anticodon interaction, translational pause sites, and/or other elements relevant to protein folding. Optimization methods known in the art may be used in the present invention, e.g., those described in WO 2021/226461, or GeneOptimizer by ThermoFisher and OptimumGene™, which is described in US 2011/0081708, the contents of which are incorporated herein by reference in its entirety.

During the reaction, the RNA polymerase synthesizes RNA in a template-dependent manner. In some embodiments, the RNA polymerase is SP6 RNA polymerase. In other embodiments, the RNA polymerase is T7 RNA polymerase. In some embodiments, the RNA polymerase is T3 RNA polymerase.

As the RNA polymerase transcribes the nucleotide sequence of the template into RNA, it incorporates ribonucleotides (NTPs) into the nascent RNA transcript. During incorporation of an NTP, pyrophosphate (PPi) is released.

In some embodiments, the IVT reaction further comprises a pyrophosphatase. The pyrophosphatase hydrolyses PPi into inorganic phosphate (Pi).

To function efficiently, the RNA polymerase requires a divalent cation as a co-factor. Suitable divalent cations include magnesium (Mg²⁺) or manganese (Mn²⁺). In some embodiments, the IVT reaction includes Mg²⁺ or Mn²⁺.

### Reactants

In some embodiments, the method of the invention is used to monitor the change in the amount of one reactant. In some embodiments, the change in the amount of more than one reactant is monitored.

The major reactants of an IVT reaction are the NTPs. NTPs are used to form the main product of the IVT reaction, the RNA. The progress of the IVT reaction can be monitored by determining changes in the amount of NTPs. Indeed, NTP levels will decrease as they are incorporated into the RNA. Accordingly, in some embodiments, the method of the invention monitors changes in the amount of one or more NTPs. In some embodiments, changes in the amount of one, two, or three NTPs is monitored simultaneously. For example, depending on the spectroscopy method chosen to obtain the spectrum in step (i) of the method of the invention, it may be possible to monitor changes in the amounts of an individual NTP of interest (e.g., ATP, CTP, GTP, or UTP). In some embodiments, the method of the invention is used to monitor changes in the amount of ATP. In some embodiments, the method of the invention is used to monitor changes in the amount of CTP. In some embodiments, the method of the invention is used to monitor changes in the amount of GTP. In some embodiments, the method of the invention is used to monitor changes in the amount of UTP. In some embodiments, the method of the invention is used to monitor changes in the amount of ATP and UTP, of ATP and CTP, of ATP and GTP, of GTP and CTP, of GTP and UTP, of UTP and CTP, of ATP, CTP, and GTP, of ATP, CTP, and UTP, or of GTP, UTP, and CTP.

The RNA may be synthesized from NTPs comprising naturally-occurring nucleosides (also referred to herein as “unmodified nucleosides”; i.e., adenosine, guanosine, cytidine, and uridine). Accordingly, the NTPs in the IVT reaction can be adenosine triphosphate (ATP), guanosine triphosphate (GTP), cytosine triphosphate (CTP), and uridine triphosphate (UTP).

In some embodiments, the RNA may be synthesized by including one or more modified nucleosides in the IVT reaction. Accordingly, one or more NTPs in the IVT reaction can be modified NTPs. Modified NTPs may include nucleoside analogues (e.g., an adenosine analogue, a guanosine analogue, a cytidine analogue, and/or a uridine analogue). In some embodiments, the modified NTP is a modified UTP (also referred to herein as UTPm), e.g., a uridine analogue such as N1-methylpseudouridine.

In some embodiments, the one or more modified NTPs comprises a nucleoside analogue selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, pseudouridine (e.g., N1-methylpseudouridine), 2-thiouridine, and 2-thiocytidine.

In some embodiments, the modified NTP comprises a nucleoside analogue selected from pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine. In some embodiments, the modified NTP comprises the nucleoside analogue N1-methylpseudouridine.

In some embodiments, the modified NTP comprises a nucleoside analogue selected from 5-aza-cytidine, 6-aza-cytidine, pseudoisocytidine, 3-methylcytidine, N⁴-acetyl cytidine, 5-formyl-cytidine, N⁴-methylcytidine, 5-methylcytidine, 5-halo cytidine (e.g., 5-iodo cytidine), 5-hydroxy methylcytidine, 1-methyl-pseudoisocytidine, pyrrolo-cytidine, pyrrolo-pseudoisocytidine, 2-thio-cytidine, 2-thio-5-methylcytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio zebularine, 2-thio-zebularine, 2-methoxy cytidine, 2-methoxy-5-methylcytidine, 4-methoxy pseudoisocytidine, 4-methoxy-1-methyl-pseudoisocytidine, lysidine, alpha-thio-cytidine, 2′-O-methylcytidine, 5,2′-O-dimethyl cytidine, N⁴-acetyl-2′-O-methylcytidine, N⁴,2′-O-dimethyl cytidine, 5-formyl-2′-O-methylcytidine, N⁴,N⁴,2′-O-trimethyl cytidine, 1-thio-cytidine, 2′-F-ara-cytidine, 2′-F cytidine, and 2′-OH-ara-cytidine. In some embodiments, the modified NTP comprises the nucleoside analogue 5-methylcytidine.

In some embodiments, the modified NTP comprises a nucleoside analogue selected from the group consisting of pseudouridine, N1-methylpseudouridine, 5-methylcytosine, 5-methoxyuridine, and any combination thereof.

In some embodiments, the IVT reaction comprises both unmodified and modified NTPs. For example, the NTPs in the IVT reaction can be adenosine triphosphate (ATP), guanosine triphosphate (GTP), cytosine triphosphate (CTP), and N1-methylpseudouridine triphosphate.

In some embodiments, changes in the amount of one or more unmodified NTPs are monitored during the IVT reaction. Modified NTP may be detected in substantially the same way as unmodified NTPs. Typically, no or minimal adjustments are needed to adapt a monitoring method of the invention for use with one or modified NTPs. Accordingly, in some embodiments, changes in the amount of one or more modified NTPs are monitored during the IVT reaction. In specific embodiments, changes in the amount of one or more unmodified NTPs and one or more modified NTPs are monitored using the method of the invention.

Dependent on the method used for obtaining a spectrum during the IVT reaction, it may be difficult to distinguish between purine nucleotides (e.g., adenine and guanine) on the one hand, and pyrimidine nucleotides (e.g., cytosine and uracil) on the other hand, given their similarity in structure and hence the resulting similarity in spectral properties. Therefore, in some embodiments, the method of the invention is used to monitor changes in purine nucleotides and/or changes in pyrimidine nucleotides. For example, in some embodiments, the changes in purine nucleotides (e.g., ATP and GTP) is monitored separately from the changes in pyrimidine nucleotides (e.g., CTP and UTP). In some embodiments, a spectrum is obtained to monitor the changes in all NTPs in the IVT reaction. Specifically, in some embodiments, the changes in ATP, UTP, GTP, and CTP are all monitored separately.

### Products

The major products of the IVT reaction can include RNA, inorganic pyrophosphate (PPi), H′, and inorganic phosphate (Pi). During the course of the IVT reaction, the amount of RNA increases. As reactants are used up during the IVT reaction, the amount of RNA may plateau. The method of the invention makes it possible to monitor the progress of the IVT reaction by determining changes in the amount of RNA. Monitoring the amount of RNA is useful because it allows an operator to terminate a reaction early, e.g., because the amount of RNA does not increase in the predicted manner during the initial stages of the IVT reaction. Moreover, monitoring the amount of RNA as the IVT reaction proceeds enables an operator to stop the IVT reaction once the amount of RNA starts plateauing, possibly shortening the time required for the IVT reaction. Both interventions can be useful to increase the output of RNA during manufacturing. Accordingly, in some embodiments, the method of the invention monitors changes in the amount of RNA throughout the IVT reaction.

During NTP incorporation, PPi and H′ can be generated as by-products of the IVT reaction. The progress of the IVT reaction can be monitored by determining changes in the amount of such by-products. In some embodiments, changes in the amount of PPi and/or H are monitored during the IVT reaction.

As a by-product of an IVT reaction, PPi can form insoluble precipitates with magnesium, which is typically included as a reagent. The incorporation of magnesium in the Mg₂PPi insoluble precipitates means that there is a reduced amount of Mg²⁺ available to act as a co-factor for RNA polymerase. Therefore, the presence of PPi in the IVT reaction is associated with reduced transcription efficiency. Accordingly, in some embodiments, changes in the amount of PPi are monitored during the IVT reaction.

In some embodiments, the IVT reaction includes a pyrophosphatase. Pyrophosphatase can hydrolyse PPi into Pi. Consequently, the amount of Mg₂PPi precipitate that is formed during the IVT reaction can be reduced. Changes in the amount of Pi during the course of the IVT reaction can provide an indirect measure of the progress of the IVT reaction. Accordingly, in some embodiments, changes in the amount of Pi are monitored during the IVT reaction.

The pH typically increases during the IVT reaction due to the generation of H′. An increase in pH can negatively affect the efficiency of the IVT reaction. Accordingly, in some embodiments, changes in the amount of H′ are monitored during the IVT reaction.

In some embodiments, the change in the amount of one product (e.g., RNA) is monitored. In some embodiments, the change in the amount of more than one product is monitored. For example, changes in the amount of RNA may be monitored at the same time as monitoring changes in the amount of other products. In some embodiments, the amount of RNA and the amount of PPi and/or Pi is monitored in the reaction vessel by acquiring a spectrum from which information about the amount of these products can be obtained.

In some embodiments, the change in the amount of one or more reactants (e.g., NTPs) and one product (e.g., RNA) is monitored. For example, in addition to the amount of RNA, the same spectrum may also provide information about the amount of NTPs (e.g., NTPs) in the IVT reaction. In some embodiments, the change in the amount of one or more reactants and one or more products (e.g., RNA and PPi and/or Pi) is monitored.

In some embodiments, the amounts of RNA and NTPs (e.g., purine and/or pyrimidine nucleotides) are monitored throughout the IVT reaction. In some embodiments, the amounts of RNA and PPi and/or Pi are monitored throughout the IVT reaction. In some embodiments, the amounts of (i) RNA, (ii) NTPs (e.g., purine and/or pyrimidine nucleotides), and (iii) PPi and/or Pi are monitored throughout the IVT reaction. In some embodiments, the amounts of (i) RNA, (ii) NTPs (e.g., purine and/or pyrimidine nucleotides), (iii) PPi and/or Pi, and optionally (iv) H′ are monitored throughout the IVT reaction.

### Additional Components

The IVT reaction typically includes a buffering agent, e.g., Tris, HEPES, citrate, acetate, or phosphate. In some embodiments, the buffering reagent is chosen on the basis that it does not interfere with the spectral readings of the reactants and/or products of interest in the IVT reaction. A suitable buffering reagent is, for example, Tris. In some embodiments, the Tris in the IVT reaction has a concentration of 10-100 mM. In some embodiments, the Tris in the IVT reaction has a concentration of 15-35 mM. In one embodiment, the Tris in the IVT reaction has a concentration of 25 mM.

In some embodiments, the IVT reaction also includes one or more salts, e.g., sodium chloride and magnesium chloride. Magnesium chloride can be used to provide the divalent cation that can act as a co-factor to increase the efficiency of the RNA polymerase. In some embodiments, the IVT reaction further comprises an RNase inhibitor. In some embodiments, the IVT reaction further comprises DTT.

### Termination

The IVT reaction can be terminated, e.g., by addition of a nuclease (e.g., DNase I) to digest the template (e.g., a DNA template). The template concentration may be monitored throughout the IVT reaction, or during the termination phase only. In some embodiments, the amount of the template (e.g., the DNA template) is monitored in the reaction vessel by acquiring a spectrum from which information about the amount of the template can be obtained. This information can be used to monitor the digestion of the template (e.g., the DNA template) after the addition of the nuclease (e.g., DNase I). The capability of monitoring termination of the IVT reaction is useful, e.g., as confirmation that a batch of mRNA is ready for the next manufacturing step (e.g., purification).

Alternatively, the template may be able to be removed from solution without undergoing degradation. For example, the template may be immobilised on magnetic beads. Monitoring the amount of the template (e.g., the DNA plasmid) would demonstrate the removal of the template, e.g., by bead removal with a magnet.

A protease (e.g., proteinase K) may be added to inactivate any enzyme components (e.g., the RNA polymerase and a nuclease, if present). DTT may also be added to quench the reaction if necessary. The concentration of the protease (e.g., proteinase K) and/or other enzyme components may be monitored throughout the IVT reaction, or during the termination phase only.

In some embodiments, the presence and/or the amount of one or more of (i) the template, (ii) the nuclease (e.g., DNase I), (iii) the protease (e.g., proteinase K), and (iv) the RNA polymerase is/are monitored in the reaction vessel by acquiring a spectrum from which information about the amount of one or more of the template, the nuclease, the protease, and the RNA polymerase can be obtained. For example, information about the presence of the nuclease or protease may be useful in an automated processes to confirm that the enzymatic component was added to the reaction vessel at the appropriate time (i.e., during the termination phase after completion of the IVT reaction) and/or to confirm that the protease has degraded enzyme components.

In some embodiments, the digestion of the template and the presence of the nuclease (e.g., DNase I) are monitored. In some embodiments, the digestion of the template (e.g., by DNase I) and the digestion of the RNA polymerase (e.g., by proteinase K) are monitored. In some embodiments, the digestion of the template is monitored followed by monitoring of the digestion of the RNA polymerase and the nuclease (e.g., DNase I) by a protease (e.g., proteinase K). In some embodiments, autodigestion of the protease (e.g., proteinase K) is monitored.

### Spectrometer

To obtain the spectrum in step (i) and the pre-determined reference spectrum in step (ii) of the method of the invention, various spectrometers can be used. For example, a light-scattering (LS)-based technique, as opposed to an absorbance-based method, may be used for implementing the claimed method. As demonstrated in the examples of the present application, a Raman spectrometer or turbidimeter can be used when working the invention. Accordingly, in some embodiments, the spectrum in step (i) and the reference spectrum in step (ii) of the method of the invention are obtained using LS-based techniques.

Suitable LS-based techniques include Raman spectroscopy. Accordingly, in some embodiments, a spectrometer for use with the method of the invention is a Raman spectrometer.

LS-based methods may be complex in terms of the equipment and the required data analysis. A relatively simple way for implementing a method of the invention involves the use of a turbidimeter. During the IVT reaction, insoluble Mg₂PPi precipitates can form as a by-product. As the IVT reaction progresses and the amount of this by-product rises, turbidity increases. The rate of emergence of turbidity provides an indirect indicator of the production of RNA, as the formation of Mg₂PPi precipitates is dependent on a by-product from an IVT reaction. Accordingly, in some embodiments, a spectrometer for use with the method of the invention is a turbidimeter. In some embodiments, assessing turbidity can provide a complementary method for monitoring the IVT reaction.

In some embodiments, a vibrational technique may be used for implementing the claimed method. Vibrational spectroscopy is based on period changes of polarizability (Raman) or dipole moment (infrared) caused by molecular vibrations of molecules, or groups of atoms within a molecule, and the discrete energy transitions and changes of frequencies during the scattering or adsorption of electromagnetic radiation. An advantage of using vibrational techniques such as Raman or infrared (IR) spectroscopy is the capacity to monitor multiple components, such as reactants and/or products, simultaneously during the IVT reaction.

As demonstrated in one of the exemplified embodiments of the invention, wavelength regions corresponding to major reactants (e.g., NTPs) and products (e.g., mRNA, PPi, and/or Pi) of an IVT reaction can be identified in a vibrational spectrum. Indeed, a software-based comparison with a pre-determined reference spectrum not only allows the quantitative analysis of reactants or products, but also the quantitative determination of a particular reactant or product with a specified timeframe as the IVT reaction proceeds in a reaction vessel.

A quantitative analysis may include determining changes of the amounts of one or more reactants or products relative to a baseline—e.g., a baseline spectrum just before the IVT reaction is initiated (e.g., by the addition of an RNA polymerase). A quantitative determination may include monitoring the concentration of one or more reactants or products during the IVT reaction.

Advantageously, with a vibrational technique such as Raman or IR spectroscopy, reactants and/or products of an IVT reaction can be monitored without the need for labelling the reactants or product or obtaining an aliquot from the reaction vessel.

Accordingly, in some embodiments, a spectrometer for use with the method of the invention is a Raman spectrometer. In some embodiments, a spectrometer for use with the method of the invention is an IR spectrometer. In some embodiments, a spectrometer for use with the method of the invention is a near-IR (NIR) spectrometer. In some embodiments, a spectrometer for use with the method of the invention is a mid-IR (MIR) spectrometer.

In some embodiments, it may be advantageous to use more than one method of spectroscopy to monitor the IVT reaction. In particular, a Raman spectrometer, an IR spectrometer (e.g., an NIR or MIR spectrometer), and a turbidimeter, or any combination of two or more thereof, may be used to monitor the IVT reaction. In some embodiments, a Raman spectrometer and a turbidimeter are used to monitor the IVT reaction. In other embodiments, an IR spectrometer and a turbidimeter are used to monitor the IVT reaction. In some embodiments, a Raman spectrometer and an IR spectrometer are used to monitor the IVT reaction.

### Obtaining Spectra

The present invention involves obtaining at least one spectrum during the course of the IVT reaction to monitor the amount of a reactant and/or product within the reaction vessel. In some embodiments, the present invention also involves obtaining at least one spectrum during the course of the IVT reaction to monitor the amount of an RNA polymerase and/or template (e.g., a DNA template) within the reaction vessel. In some embodiments, the present invention also involves obtaining at least one spectrum during the course of the IVT reaction to monitor the addition of a nuclease (e.g., DNase I) or protease (proteinase K) within the reaction vessel to quench or terminate the IVT reaction.

In some embodiments, a spectrum comprising information about one or more reactants (e.g., one or more NTPs) is obtained during the course of the IVT reaction. In some embodiments, a spectrum of one or more products (e.g., RNA) is obtained during the course of the IVT reaction.

The acquisition time of a spectrum is related to the width of the wavelength region, i.e., obtaining a spectrum of a wider wavelength region takes longer than that of a narrower wavelength region. In order to reduce the acquisition time of the spectrum, it may be preferable to limit the step of obtaining a spectrum to a wavelength region specific to one particular reactant or product. For instance, in some embodiments, the spectrum obtained in step (i) of the method of the invention is limited to a wavelength region that provides information about the amount of RNA present in the reaction vessel. Reduction of the acquisition time by reducing the wavelength region allows monitoring a reactant or product essentially in real time. Accordingly, in some embodiments, a Raman spectrum of a wavelength region comprising 600 cm⁻¹ to 1300 cm⁻¹ is obtained. In some embodiments, a Raman spectrum of a wavelength region comprising 800 cm⁻¹ to 1250 cm⁻¹ is obtained.

In some embodiments, it may be desirable to obtain a spectrum that covers multiple reactants and products of the IVT reaction. Accordingly, in some embodiments, the spectrum obtained in step (i) spans a wavelength region suitable for monitoring the global evolution of multiple reactants and products during the IVT reaction. In some embodiments, a wavelength region is selected that provides information about the amounts of (i) RNA, (ii) NTPs (e.g., purine and/or pyrimidine nucleotides), (iii) PPi and/or Pi, and optionally (iv) H+ in the IVT reaction. For example, the inventors found that acquiring a Raman spectrum of a wavelength region comprising 150 cm⁻¹ to 4000 cm⁻¹ required 50 seconds and provided information about all the aforementioned products and reactants.

In some embodiments, each spectrum in a series is obtained over a time period of less than one minute. In some embodiments, each spectrum in a series is obtained over a time period of less than 60 seconds, e.g., less than 50 seconds, less than 30 seconds, less than 20 seconds, less than 10 seconds, or less than 5 seconds. In some embodiments, each spectrum in a series is obtained over a time period of 1 to 60 seconds, e.g., 10-50 seconds or 5-20 seconds.

The acquisition of a spectrum, or spectra, during the course of the IVT reaction can provide qualitative and/or quantitative data to inform the user of the reaction progress. The term ‘information’ may be used to describe such qualitative and/or quantitative data acquired. In some embodiments, a spectrum comprising information about one or more reactants and one or more products is obtained during the course of the IVT reaction. For example, it may be advantageous to obtain a spectrum across a wider wavelength region that includes information about multiple reactants and products of the IVT reaction, in particular when optimizing the reaction conditions for the production of a particular RNA, e.g., to account for the nucleotide composition and/or length of the RNA.

In some embodiments, a series of spectra are obtained. By obtaining multiple spectra, the data can be merged to reduce background noise. Optimization of the signal-to-noise ratio helps to better distinguish the signal from the background noise associated with any detection method. In some embodiments, the series of spectra comprises a set of a least 3, at least 5, or at least 9 spectra. In some embodiments, a series of 3 to 20 spectra are obtained. In some embodiments, a series of 5 to 15 spectra are obtained. In some embodiments, a series of 8 to 12 spectra are obtained.

An important consideration when obtaining spectra is the impact of acquisition on spectral resolution. Whilst obtaining fewer spectra can reduce the acquisition time, this may result in a loss of quality. For example, the noise of the spectra may not be as easily distinguished from the signal and may therefore affect spectral interpretation and analysis. The number of spectra that may be acquired to obtain a workable data set may also depend on the particular spectroscopic method and/or the particular spectrometer used for obtaining the spectrum in step (i) of the method of the invention. For example, the inventors found that obtaining a series of 10 Raman spectra reduced background noise. Accordingly, in a specific embodiment, a series of 10 spectra are obtained. In some embodiments, each spectrum in a series is obtained over a time period of 1 to 60 seconds, e.g., 10-50 seconds.

Upon initiation of the IVT reaction, the amount of the one or more reactants and the one or more products may change throughout the course of the reaction until the reaction has been terminated, e.g., by addition of a nuclease (e.g., DNase I) to digest the template. Accordingly, in some embodiments, a series of spectra are obtained over the course of the IVT reaction. It may be desirable to obtain spectra at intervals during the course of the IVT reaction, e.g., to determine the evolution of the amounts of one or more reactants and/or one or more products. In some embodiments, a series of spectra are obtained over the course of the IVT reaction, typically at equally spaced intervals. In some embodiments, multiple series of spectra are obtained over the course of the IVT reaction in order to reduce the signal to noise ratio at individual data points.

In some embodiments, a spectrum or series of spectra is obtained at intervals of 1-30 minutes over the course of the IVT reaction. In some embodiments, a spectrum or series of spectra is obtained at 30-minute intervals. In some embodiments, a spectrum or series of spectra is obtained at 15-minute intervals. In some embodiments, a spectrum or series of spectra is obtained at 10-minute intervals. In some embodiments, a spectrum or series of spectra is obtained at 5-minute intervals. In some embodiments, a spectrum or series of spectra is obtained at 4-minute intervals. In some embodiments, a spectrum or series of spectra is obtained at 3-minute intervals. In some embodiments, a spectrum or series of spectra is obtained at 2-minute intervals. In some embodiments, a spectrum or series of spectra is obtained at 1-minute intervals.

In some embodiments, a spectrum or series of spectra is obtained at intervals of less than 1 minutes, e.g., at intervals of 50 seconds or less than, 40 seconds or less, 30 seconds or less, 20 seconds or less, or 10 seconds or less.

In some embodiments, spectra are obtained continuously over the course of the IVT reaction. The obtained spectra may be combined into sets of spectra as described above to improve the signal to noise ratio for individual data points. For example, 3, 4, 5, 6, 7, 8, 9 or 10 spectra may be obtained and combined into a set. The set of spectra may be represented as a single data point although they represent a series of spectra acquired over a certain period of time (e.g., over a period of 1, 2, 3, 5, 6, 7, 8, 9 or 10 minutes).

### Monitoring Reactants and or Products During the IVT Reaction

In some embodiments, a spectrum is obtained for a wavelength region to monitor the global evolution of the amount of at least one reactant (e.g., one or more NTPs) and at least one product (e.g., RNA) of the IVT reaction.

In some embodiments, the wavelength region provides information for all the major products and reactants of the IVT reaction, including RNA, PPi/Pi, and NTPs. A suitable wavelength region to monitor these products and reactants using a Raman spectrometer comprises 300 cm⁻¹ to 3000 cm⁻¹. In some embodiments, the wavelength region comprises 600 cm⁻¹ to 1300 cm⁻¹. In other embodiments, the wavelength region comprises 800 cm⁻¹ to 1250 cm⁻¹.

In some embodiments, the spectrum obtained in step (i) of the method of the invention spans a wavelength region specific to a single product or reactant of the IVT reaction.

In some embodiments, the product is RNA (e.g., mRNA). In some embodiments, the amount of RNA is monitored using a Raman spectrometer. A suitable wavelength region to monitor the amount of RNA in an IVT reaction using a Raman spectrometer comprises 801 cm⁻¹ to 831 cm⁻¹. In some embodiments, the amount of RNA in an IVT reaction is monitored at a wavelength of about 810 cm⁻¹.

In some embodiments, the product is Pi. In some embodiments, the amount of Pi is monitored using a Raman spectrometer. A suitable wavelength region to monitor the amount of Pi in an IVT reaction using a Raman spectrometer comprises 875 cm⁻¹ to 900 cm⁻¹.

In some embodiments, the reactant is one or more NTPs (e.g., one, two or three individual NTPs). In some embodiments, the reactant comprises two or more NTPs (e.g., purine nucleotides or pyrimidine nucleotides). In some embodiments, the amount of one or more NTPs is monitored using a Raman spectrometer. A suitable wavelength region to monitor the amount of NTPs in an IVT reaction using a Raman spectrometer comprises 600 cm⁻¹ to 1300 cm⁻¹, including, e.g., 700 cm⁻¹ to 800 cm⁻¹. In some embodiments, a narrower wavelength region is used for monitoring the amount of NTPs in the IVT reaction.

In some embodiments, a suitable wavelength region to monitor the amount of NTPs in an IVT reaction using a Raman spectrometer comprises 1100 cm⁻¹ to 1120 cm⁻¹ or 1107 cm⁻¹ to 1146 cm⁻¹. The wavelength region of 1113 cm⁻¹ to 1115 cm⁻¹, in particular, corresponds to the stretching of PO₂⁻ in the IVT reaction. Accordingly, in some embodiments, a suitable wavelength region to monitor the amount of NTPs in an IVT reaction using a Raman spectrometer comprises 1113 cm⁻¹ to 1115 cm⁻¹ (e.g., 1115 cm⁻¹).

NTPs can be categorized by the presence of a purine or pyrimidine base. In some embodiments, wavelength regions are selected that allow the separate detection of purine nucleotides (e.g., ATP and GTP) and pyrimidine nucleotides (e.g., CTP and UTP).

In some embodiments, the reactant is a purine nucleotide. In some embodiments, the amount of purine nucleotides is monitored using a Raman spectrometer. For example, using a Raman spectrometer, GTP and ATP can also be detected in a wavelength region comprising 1300 cm⁻¹ to 1600 cm⁻¹. Accordingly, in some embodiments, a suitable wavelength region to monitor the amount of purine nucleotides in an IVT reaction using a Raman spectrometer comprises 1300 cm⁻¹ to 1600 cm⁻¹. In some embodiments, a suitable wavelength region to monitor the amount of GTP and ATP in an IVT reaction using a Raman spectrometer comprises 1580 cm⁻¹. The wavelength of 1580 cm⁻¹ corresponds to the stretching of the C═N bond. Accordingly, in some embodiments, a suitable wavelength region to monitor the amount of GTP and ATP in an IVT reaction using a Raman spectrometer comprises 1580 cm⁻¹.

The primary Raman shift in the wavelength region comprising 1560 cm⁻¹ to 1580 cm⁻¹ is due to the amount of GTP in an IVT reaction. Therefore, in some embodiments, a suitable wavelength region to monitor the amount of GTP in an IVT reaction using a Raman spectrometer comprises 1560 cm⁻¹ to 1600 cm⁻¹, e.g., 1560 cm⁻¹ to 1590 cm⁻¹ or 1560 cm⁻¹ to 1580 cm⁻¹.

Moreover, distinct peaks for GTP and ATP in the Raman spectrum can be identified in the wavelength region comprising 650 cm⁻¹ to 750 cm⁻¹. A suitable wavelength region to monitor the amount of GTP in an IVT reaction using a Raman spectrometer comprises 650 cm⁻¹ to 700 cm⁻¹. A suitable wavelength region to monitor the amount of ATP in an IVT reaction using a Raman spectrometer comprises 700 cm⁻¹ to 750 cm⁻¹. In some embodiments, both the wavelength region comprising 650 cm⁻¹ to 750 cm⁻¹ and the wavelength region comprising 1560 cm⁻¹ to 1580 cm⁻¹ are monitored with Raman spectrometer to determine the amounts of ATP and GTP.

In some embodiments, the reactant is a pyrimidine nucleotide. In some embodiments, the amount of pyrimidine nucleotides is monitored using a Raman spectrometer. Using a Raman spectrometer, CTP can be detected at 770 cm⁻¹ to 785 cm⁻¹, e.g., at 780 cm⁻¹, which corresponds to the cytosine ring vibrations. UTP (including modified UTP) can be detected at the wavelength region of 785 cm⁻¹ to 810 cm⁻¹, e.g., at 786 cm⁻¹ to 789 cm⁻¹ due to the stretching of the C—C bond, corresponding to carbon 5 and 6 of uracil. In one embodiment, a suitable wavelength region to monitor the amount of pyrimidine nucleotides in an IVT reaction using a Raman spectrometer comprises 780 cm⁻¹ to 789 cm⁻¹.

Alternatively, or in addition, CTP-UTP can also be detected at 1220 cm⁻¹ to 1250 cm⁻¹, e.g., 1230 cm⁻¹ to 1245 cm⁻¹ due to the stretching of the C═N bond. Accordingly, in some embodiments, a suitable wavelength region to monitor the amount of pyrimidine nucleotides in an IVT reaction using a Raman spectrometer comprises 1230 cm⁻¹ to 1245 cm⁻¹.

Using a Raman spectrometer, CTP can be detected by monitoring at 1240 cm⁻¹ and 1290 cm⁻¹. The 1240 cm⁻¹ wavelength corresponds to C—N stretching of the unsaturated amine. Accordingly, in some embodiments, a suitable wavelength region to monitor the amount of CTP in an IVT reaction using a Raman spectrometer comprises 1240 cm⁻¹ and 1290 cm⁻¹.

In some embodiments, the spectrum obtained in step (i) of the method of the invention is measured at a wavelength specific to a single product or reactant of the IVT reaction. For example, using a Raman spectrometer, ATP can be detected at 633 cm⁻¹, which corresponds to vibration of the purine ring. Using a Raman spectrometer, a suitable wavelength region to monitor the amount of UTP comprises 1670 cm⁻¹, which is associated with carbon 4 ketone function. Accordingly, in some embodiments, a suitable wavelength to monitor the amount of ATP and UTP in the IVT reaction using a Raman spectrometer is 633 cm⁻¹ and 1670 cm⁻¹, respectively.

Mg₂PPi is a by-product of the IVT reaction and accumulates as an insoluble precipitate. The amount of Mg₂PPi can be detected by monitoring the turbidity of the solution in which the IVT reaction. Turbidity can be monitored using a turbidimeter, a UV spectrometer, or a nephelometer. It can be measured in a wavelength region comprising 290 nm to 410 nm, e.g., 300 nm to 350 nm. Exemplary wavelengths for measuring turbidity using a UV spectrometer are 310 nm, 320 nm, 330 nm, 340 nm or 350 nm. For example, using a UV spectrometer, turbidity can be measured at an absorbance of 320 nm. The presence of insoluble components can also be measured using a turbidimeter (the measurements are typically given in nephelometric turbidity units [NTUs]).

### Monitoring Termination of the IVT Reaction

In some embodiments, a spectrum is obtained for a wavelength region to monitor the template and RNA polymerase during the IVT reaction. For example, changes in the amount of the template or the RNA polymerase may be indicative of contamination of the IVT reaction.

In some embodiments, the template (e.g., the DNA template) is monitored throughout the IVT reaction. In some embodiments, the template is monitored only during the termination phase, e.g., to confirm destruction or removal of the template. In some embodiments, the template is monitored using a Raman spectrometer. A suitable wavelength region to monitor the template using a Raman spectrometer is 505 cm⁻¹ to 705 cm⁻¹. In some embodiments, a suitable wavelength region to monitor the template using a Raman spectrometer is 1325 cm⁻¹ to 1365 cm⁻¹ or 1585 cm⁻¹ to 1720 cm⁻¹.

In some embodiments, the RNA polymerase (e.g., SP6 RNA polymerase) is monitored throughout the IVT reaction. In some embodiments, the RNA polymerase is monitored during the termination phase only to confirm destruction of the enzyme (e.g., by protease digestion). In some embodiments, the RNA polymerase is monitored using a Raman spectrometer. A suitable wavelength region to monitor an RNA polymerase such as SP6 RNA polymerase using a Raman spectrometer is 780 cm⁻¹ to 1200 cm⁻¹. In some embodiments, a suitable wavelength region to monitor an RNA polymerase such as SP6 RNA polymerase using a Raman spectrometer is 1200 cm⁻¹ or 1430 cm⁻¹ to 1510 cm⁻¹.

In some embodiments, a spectrum is obtained for a wavelength region to monitor the addition of components (e.g., enzymatic components) to terminate the IVT reaction. In an automated system, confirmation of the addition of the enzymatic components at the correct timepoint of the IVT reaction can be useful.

In some embodiments, the addition or amount of a nuclease (e.g., DNase I) is monitored. In some embodiments, the amount of the nuclease (e.g., DNase I) is monitored during the termination phase only to confirm destruction of the enzyme (e.g., by proteinase K digestion). In some embodiments, the addition or amount of the nuclease (e.g., DNase I) is monitored using a Raman spectrometer. A suitable wavelength region to monitor a nuclease such as DNase I using a Raman spectrometer is 450 cm⁻¹ to 520 cm⁻¹. In some embodiments, a suitable wavelength region to monitor DNase I using a Raman spectrometer is 1000 cm⁻¹ to 1090 cm⁻¹ or 2915 cm⁻¹ to 3000 cm⁻¹.

In some embodiments, the addition of a protease (e.g., proteinase K) is monitored. In some embodiments, the addition of the protease is monitored using a Raman spectrometer. A suitable wavelength region to monitor a protease such as proteinase K using a Raman spectrometer is 505 cm⁻¹ to 610 cm⁻¹, e.g., 550 cm⁻¹ to 600 cm⁻¹. In some embodiments, a suitable wavelength region to monitor a protease such as proteinase K using a Raman spectrometer is 715 cm⁻¹ to 775 cm⁻¹ or 1385 cm⁻¹ to 1395 cm⁻¹.

### Pre-Processing Spectra

Raw spectral data obtained in step (i) of a method of the invention may be processed to improve the signal to noise ratio. Accordingly, in some embodiments, a spectrum obtained in step (i) is pre-processed prior to step (ii). In some embodiments, each of the spectra obtained in the series are pre-processed prior to step (ii). Pre-processing can facilitate the comparative analysis of spectra. For example, a wavelength region of a spectrum obtained in step (i) may be normalized relative to a wavelength region with low or no background noise. Using a Raman spectrometer, a suitable wavelength region with low background noise comprises 3100 cm⁻¹ and 3600 cm⁻¹. Normalization is typically done by employing a suitable algorithm, e.g., a Standard Normal Variate (SNV) algorithm. In some embodiments, a spectrum or a series of spectra obtained in step (i) is/are normalized to a suitable wavelength region. In one embodiment, the spectrum or series of spectra is/are acquired with a Raman spectrometer and a wavelength region of 3100 cm⁻¹ to 3600 cm⁻¹ is used for normalization.

Alternatively, or in addition, pre-processing may apply a digital filter to smooth the data using a series of spectra. Applying such a filter increases the precision of the data without distorting the signal tendency. In some embodiments, the digital filter fits successively acquired spectra comprising adjacent data points with a low-degree polynomial by the method of linear least squares. In a typical embodiment, a series of spectra is obtained in equally spaced intervals. As the data points are therefore equally spaced, an analytical solution to the least-squares equations can be found in the form of a single set of “convolution coefficients” that can be applied to all spectra in the series to provide a smoothed data set. For example, a suitable digital filter may employ a Savitzky-Golay (SG) algorithm.

### Comparing a Spectrum to a Reference Spectrum

In accordance with a method of the invention, a spectrum of a reactant or product obtained in step (i) is compared to a pre-determined reference spectrum of the reactant or product in step (ii).

In some embodiments, the pre-determined reference spectrum is the first spectrum, or a first series of spectra, obtained during the IVT reaction. In these embodiments, the first spectrum or series of spectra serves as a baseline of the IVT reaction. Any change in the amount of a reactant or product of interest reflects the progress of the IVT reaction.

In some embodiments, the pre-determined reference spectrum is a spectrum or series of spectra obtained during an IVT reaction using known conditions and reactants. In some embodiments, the pre-determined reference spectrum is a spectrum or series of spectra of a reactant or product of the IVT reaction, typically obtained with the reactant or product dissolved or suspended in the reaction buffer used for the IVT reaction. In either scenario, the concentration of a reactant and/or product is known for the pre-determined reference spectrum and therefore can be used to infer the concentration of the reactant and/or product of the spectrum or series of spectra obtained in step (i) of a method of the invention.

Accordingly, in some embodiments, the pre-determined reference spectrum correlates to a specified concentration of a reactant or product. In some embodiments, the pre-determined reference spectrum is used to determine the concentration of a reactant or product. In some embodiments, a series of pre-determined reference spectra are used to calculate the concentration of a reactant or product wherein each spectrum of the series correlates to a different concentration.

In some embodiments, determining the concentration of the reactant or product comprises linear regression analysis. In some embodiments, a partial least square (PLS) model is used to determine the concentration of the reactant or product from a series of pre-determined reference spectra, wherein each spectrum of the series correlates to different concentrations of the reactant or product.

In some embodiments, the concentration of a reactant or product is determined using linear regression analysis. In one embodiment, a PLS model is used to determine the concentration of a reactant or product. PLS finds a linear regression model by projecting the predicted variables and the observable variables to a new space. The fundamental relationships are set between the variables in this new space and projected afterwards back into the original space. PLS regression is widely used in chemometrics and related areas.

In some embodiments, step (ii) comprises a qualitative spectral comparison between the spectrum or series of spectra obtained in step (i) and the pre-determined reference spectrum. In some embodiments, a qualitative spectral comparison comprises calculating a weighted spectral difference (WSD) value between the spectrum or series of spectra obtained in step (i) and the pre-determined reference spectrum.

In some embodiments, a WSD value within one standard deviation or less indicates that there is a high similarity, e.g., no significant difference, between the spectrum obtained in step (i) and the pre-determined reference spectrum. Accordingly, in these embodiments, the amount of the reactant or product of the spectrum obtained in step (i) is considered to be the same as the amount of the reactant or product of the pre-determined reference spectrum, or the amount of the reactant or product of the spectrum obtained in step (i) is considered to remain unchanged relative to the amount of the reactant or product of the pre-determined reference spectrum, as applicable.

In some embodiments, a WSD value of more than one standard deviation (e.g., two standard deviations) indicates that there is low similarity, e.g., a significant difference, between the spectrum obtained in step (i) and the pre-determined reference spectrum. Accordingly, in these embodiments, the amount of the reactant or product of the spectrum obtained in step (i) is considered to be greater or lower than the amount of the reactant or product of the reference spectrum, as applicable.

### Process Optimization

In some embodiments, the methods of the invention can be used to optimize reaction conditions prior to the large-scale manufacturing of an RNA (e.g., mRNA). For example, obtaining a series of spectra by monitoring an IVT reaction can be used to create a kinetic model for one or more reactants and/or products. The kinetic model can be employed to predict or simulate changes in the amount of the one or more products under different conditions, e.g., at different starting concentrations of the one or more reactants. Using a kinetic model therefore may reduce the number of experiments that need to be performed to identify an optimal set of conditions to produce an RNA using an IVT reaction.

In some embodiments, a kinetic model is used to predict the yield of RNA produced from an IVT reaction. In some embodiments, a kinetic model is used to simulate the IVT reaction to predict the consumption of a reactant. In specific embodiments, a kinetic model is used to predict the consumption of NTPs during an IVT reaction.

The kinetic model may also be used as reference to which a spectrum obtained during an IVT reaction is compared in order to determine whether the IVT reaction proceeds at the expected level of efficiency. Typically, the kinetic model is created with a series of spectra obtained from an IVT reaction that was performed at similar, or identical, conditions, to the IVT reaction that is monitored. In some embodiments, a kinetic model is used to predict the yield of a product of the IVT reaction (e.g., RNA).

### Manufacturing RNA

The monitoring methods of the invention also find use as means of process control or quality control during the manufacturing of RNA, in particular during the large-scale production of RNA. For example, the monitoring methods of the invention may be particularly useful in the large-scale production of RNA, such as mRNA, for use in therapeutic applications.

Accordingly, the invention also relates to a method of manufacturing an RNA (e.g., an mRNA) using an in vitro transcription (IVT) reaction, said method comprising: (a) providing a DNA template comprising the nucleotide sequence of the RNA operationally linked to an RNA polymerase promoter; (b) adding the DNA template to a reaction vessel comprising an RNA polymerase and necessary reactants to initiate the IVT reaction; and (c) monitoring one or more of the reactants or products of the IVT reaction using a method of monitoring the IVT reaction described herein. In some embodiments, the one or more reactants or products is RNA.

The invention further relates to a method of manufacturing an RNA (e.g., an mRNA) using an in vitro transcription (IVT) reaction comprising: (a) monitoring the production of the RNA within a reaction vessel by (i) obtaining a spectrum of the RNA during the IVT reaction to determine a first value, and (ii) comparing the first value obtained in step (i) to a second value derived from a pre-determined reference spectrum of RNA; and (b) purifying the RNA if the first value is equal to, or exceeds, the second value.

### Quality Control

The monitoring of an IVT reaction through a spectroscopic method as described herein, e.g., Raman spectroscopy, can be used to determine whether an IVT reaction during manufacturing of a batch of RNA (e.g., mRNA) meets one or more pre-determined parameters. The decision to continue or terminate the IVT reaction may be conditional on the one or more pre-determined parameters.

In some embodiments, one or more of the reactants or products are monitored throughout the course of the IVT reaction during the manufacture of RNA (e.g., mRNA). In some embodiments, the one or more reactants are NTPs. In some embodiments, the one or more products can be RNA, PPi, and/or H⁺. In some embodiments, the one or more reactants and the one or more products are monitored during the method of RNA manufacture.

For example, a spectrum of the one or more reactants or products obtained in step (i) of a monitoring method of the invention can be compared to a pre-determined reference spectrum of the one or more of the reactants or products. If the obtained spectrum sufficiently corresponds to the pre-determined reference spectrum, the IVT reaction can proceed. If not, it may be terminated.

Comparing one or more spectra obtained during the production of a batch of RNA (e.g., mRNA) to a pre-determined reference spectrum can provide confirmation that the reaction is progressing efficiently and within a set of pre-determined parameters corresponding the one or more reactants (e.g., NTPs) and/or one or more products (e.g., RNA). In some embodiments, a series of spectra are obtained at one or more specified time points during the IVT reaction wherein said series of spectra is compared to a pre-determined reference spectrum. This comparison between the series of spectra between the one or more specified time points may be used to, e.g., determine the amount, or change in the amount, of RNA.

The monitoring of multiple time points throughout the reaction can provide an insight into how the IVT reaction is performing. For example, it can be used to confirm whether the amount of RNA is increasing as expected at various intervals during the IVT reaction. In some embodiments, the two or more specified time points are at equally-spaced intervals throughout the IVT reaction. In some embodiments, each interval is 30 minutes or less, 20 minutes or less, 5 minutes or less, 2 minutes or less, 1 minute or less, or 30 seconds or less.

In some embodiments, the rate of RNA production during the IVT reaction can be measured at different time points. For example, in some embodiments, the rate of RNA production is measured at an early phase of the reaction i.e., during the first 20 minutes. In some embodiments, the rate of RNA production is measured at a later phase of the reaction i.e., after 20 minutes. In some embodiments, the rate of RNA production is measured after 30 minutes. In some embodiments, the rate of RNA production is measured after 45 minutes. In some embodiments, the amount of RNA is measured after 60 minutes. In some embodiments, the amount of RNA is measured after 75 minutes. In some embodiments, it may be useful to continuously monitor the rate of RNA production throughout the course of the IVT reaction and terminate the reaction once the rate of RNA production is about zero (meaning that the amount of RNA in the IVT reaction has plateaued).

In some embodiments, a spectral comparison may suffice to determine whether an IVT reaction during the production of a batch of RNA (e.g., mRNA) is performing as expected. For example, if a spectrum obtained during an IVT reaction significantly diverges from the pre-determined reference spectrum, the IVT reaction may not be performing efficiently and therefore the operator may take the decision to terminate it. If the spectrum from the IVT reaction is sufficiently similar to a pre-determined reference spectrum, the operator may proceed with the production run.

In some embodiments, a spectrum obtained from the IVT reaction significantly diverges from the pre-determined reference spectrum, e.g., the obtained spectrum diverges by more than one standard deviation from the pre-determined reference spectrum. In such embodiments, the operator may determine that the batch of RNA (e.g., mRNA) resulting from the IVT reaction is to be discarded.

In some embodiments, the spectrum from the IVT reaction significantly diverges from the pre-determined reference spectrum e.g., the obtained spectrum diverges by more than one standard deviation from the pre-determined reference spectrum. In such embodiments, the operator may determine that the reaction is to be terminated.

In some embodiments, the spectrum obtained from the IVT reaction is compared to a kinetic model previously determined for an IVT reaction using similar, or identical, conditions and reactants. In some embodiments, the spectrum from the IVT reaction is sufficiently similar to the kinetic model, e.g., the obtained spectrum diverges less than one standard deviation from the kinetic model. In such embodiments, the operator may determine that the IVT reaction can proceed and/or release the batch of RNA (e.g., mRNA) for subsequent production steps, e.g., purification of the RNA. In some embodiments, the spectrum from the IVT reaction significantly diverges from the kinetic model e.g., the obtained spectrum diverges more than one standard deviation from the kinetic model. In such embodiments, the operator may determine that the reaction is to be terminated and the batch of RNA (e.g., mRNA) is not to proceed to subsequent production steps.

In some embodiments, divergence from a pre-determined reference spectrum is assessed using a qualitative spectral comparison. In some embodiments, a qualitative spectral comparison comprises calculating a weighted spectral difference (WSD) value between the spectrum or series of spectra obtained in step (i) of the method of the invention and the pre-determined reference spectrum.

In some embodiments, a WSD value within one standard deviation or less indicates that there is no significant difference between the spectrum obtained in step (i) and the pre-determined reference spectrum. In some embodiments, a WSD value of more than one standard deviation (e.g., two standard deviations) indicates that there is a significant difference between the spectrum obtained in step (i) and the pre-determined reference spectrum.

In some embodiments, the spectrum obtained in step (i) of the method of the invention is used to calculate the amount of RNA present in the reaction vessel as part of the comparison with a pre-determined reference spectrum in step (ii). In some embodiments, step (i) comprises obtaining a series of spectra at one or more specified time points during the IVT reaction and step (ii) comprises determining the amount, or change in the amount, of RNA between the one or more specified time points by comparing the series of spectra and the pre-determined reference spectrum.

In some embodiments, the IVT reaction may be terminated if the RNA does not reach a target amount at the one or more specified time points.

In some embodiments, it may be advantageous to monitor the changes in the amount of RNA rather than estimating the amount (or concentration) of the RNA in the reaction vessel. In some embodiments, the IVT reaction may be terminated if the change in the amount of RNA is less than a pre-determined value at two or more specified time points. Termination in this context may mean either that the IVT reaction is aborted early, or that the IVT reaction is completed.

For instance, in some embodiments, the batch of RNA (e.g., mRNA) resulting from the IVT reaction may be discarded if the change in the amount of RNA is less than a pre-determined value at two or more specified time points. Typically, these time points are early in the IVT reaction, e.g., within the first 5-30 minutes of a production run. In some embodiments, the batch of RNA resulting from the IVT reaction may be discarded if the change in the amount of RNA is about zero between at least two or more time points. In some embodiments, the batch of RNA resulting from the IVT reaction may be discarded if the change in the amount of RNA is about zero between at least two or more time points which are at least 5, 10, 15, 20, or 25 minutes apart, e.g., about 6 minutes apart, about 7 minutes apart, about 8 minutes apart, about 9 minutes apart, about 10 minutes apart, about 15 minutes apart, about 20 minutes apart, or about 25 minutes apart.

For example, the two or more specified time points may be during the first 10 minutes, first 20 minutes, or first 30 minutes of the IVT reaction. The pre-determined value may correspond to the expected fold increase in the amount of RNA during the first 10 minutes, first 20 minutes or first 30 minutes of the IVT reaction, e.g., a 2-fold increase between the first 5 minutes and the first 20 minutes of the IVT reaction, or a 3-fold increase between the first 5 minutes and the first 30 minutes of the IVT reaction.

In some embodiments, the batch of RNA (e.g., mRNA) resulting from the IVT reaction may be discarded if the RNA does not reach a target amount or concentration at the one or more specified time points. For example, the one or more specified time points may be during the first 10 minutes, first 20 minutes or first 30 minutes of the IVT reaction. The pre-determined value may correspond to the expected concentration of the RNA during the first 10 minutes, first 20 minutes or first 30 minutes of the IVT reaction, e.g., a concentration of 1 g/L after the first 5 or 10 minutes, a concentration of 2 g/L after the first 10-20 minutes, or a concentration of 3 g/L after the first 25-30 minutes. Alternatively, one or more specified time points may be during the last third of a normal production, e.g., between 60 minutes and 90 minutes after the start of the IVT reaction. The pre-determined value may correspond to the expected concentration of the RNA during this time period, e.g., 4-5 g/L.

The spectral data obtained during the IVT reaction can also be used to determine when the amount of RNA has been plateaued. In some embodiments, this information may be used to terminate the IVT reaction and proceed to the next production step (e.g., purification).

In some embodiments, the IVT reaction may be terminated if the change in the amount of RNA is about zero between at least two or more time points. Typically, these time points are late in the IVT reaction, e.g., within 60-90 minutes of a production run. In some embodiments, the IVT reaction may be terminated if the change in the amount of RNA is about zero between at least two or more time points which are at least about 5 minutes apart, e.g., about 6 minutes apart, about 7 minutes apart, about 8 minutes apart, about 9 minutes apart, or about 10 minutes apart. For example, the IVT reaction may be terminated if there is no change in the amount of RNA between 65 minutes and 70 minutes after production was initiated (e.g., by addition of the RNA polymerase).

The invention also relates to a method of manufacturing an RNA (e.g., an mRNA) using an in vitro transcription (IVT) reaction comprising: (a) monitoring the production of the RNA within a reaction vessel by (i) obtaining a spectrum of the RNA during the IVT reaction to determine a first value and (ii) comparing the first value obtained in step (i) to a second value derived from a pre-determined reference spectrum of RNA and (b) purifying the RNA if the first value is equal to, or exceeds, the second value.

To determine the RNA yield, a pre-determined reference value may be derived from a pre-determined reference spectrum of known concentration. In some embodiments, the second value corresponds to a target concentration. In some embodiments, the target concentration of RNA is at least 3 g/L, at least 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L.

Obtaining a spectrum corresponding to RNA and comparing to a pre-determined reference spectrum at multiple time intervals can provide the information as to how the concentration of RNA is increasing during the IVT reaction. This information may be useful in determining when to terminate the reaction and purify the RNA (e.g., an mRNA for use in therapeutic applications) for downstream production steps (e.g., formulation, lyophilisation, and/or encapsulation in lipid nanoparticles). Accordingly, in some embodiments, obtaining a spectrum of the RNA to determine a first value, and comparing the first value with a second value derived from a pre-determined reference spectrum is repeated at equally spaced intervals. In some embodiments, a first value is considered equal to a second value derived from a pre-determined reference spectrum of RNA if the values are within one standard deviation of each other. In some embodiments, the RNA is purified if the first value obtained at each interval is equal to or exceeds the second value at a corresponding interval derived from the pre-determined reference spectrum.

In some embodiments, the first and second values are compared using a qualitative spectral comparison. In some embodiments, a qualitative spectral comparison comprises calculating a weighted spectral difference (WSD) value between the spectrum of the RNA obtained during the IVT reaction and the pre-determined reference spectrum.

In some embodiments, a WSD value within one standard deviation or less indicates that there is no significant difference between the spectrum obtained in step (i) and the pre-determined reference spectrum. In some embodiments, a WSD value of more than one standard deviation (e.g., two standard deviations) indicates that there is a significant difference between the spectrum obtained in step (i) and the pre-determined reference spectrum.

If the first value is not equal or exceeding the second value, the operator may determine that the batch of RNA (e.g., mRNA) does not proceed to purification.

### Process Control

In some embodiments, the information obtained from monitoring the reactants and/or products of the IVT reaction may be used to replenish the reactants in the reaction vessel. For example, during the course of the IVT reaction, NTPs are consumed. If the amount of one or more NTPs is too low, the IVT reaction is unable to proceed. For example, monitoring the amount of one or more NTPs during the course of the IVT reaction may be used to alert an operator or an operating system of the reaction vessel when said NTP(s) are reaching depletion. Therefore, the IVT reaction can be supplemented with one or more NTPs as required to maintain a suitable concentration of each NTP. In some embodiments, the IVT reaction is supplemented with one or more NTPs when the concentration of said NTP(s) approaches depletion.

In some embodiments, the IVT reaction is supplemented with one or more NTPs at least once during the course of manufacturing RNA (e.g., mRNA). In some embodiments, the IVT reaction is supplemented periodically with one or more NTPs during the course of manufacturing the RNA. In some embodiments, the IVT reaction is supplemented continuously with one or more NTPs during the course of manufacturing the RNA (e.g., during fed-batch operation of a suitably configured reaction vessel).

In some embodiments, the IVT reaction is supplemented with NTPs to maintain the concentration of NTPs that was present when the IVT reaction was initiated. In some embodiments, the IVT reaction is supplemented with NTPs to restore the concentration of NTPs to the concentration that was present when the IVT reaction was initiated. In some embodiments, the concentration of each NTP is maintained within a range of 20-100%, 20-75%, or 25%-50% that was present when the IVT reaction was initiated.

In some embodiments, the IVT reaction is supplemented with NTPs when the concentration of NTPs in the reaction is no more than 5% of the initial concentration of NTPs when the IVT reaction was initiated. In some embodiments, the IVT reaction is supplemented with one or more NTPs when the concentration of an NTP in the reaction is about 5 mM or less. In some embodiments, the IVT reaction is supplemented with one or more NTPs when the concentration of an NTP in the reaction is about 3 mM or less.

In some embodiments, each NTP is present at a concentration of 1-10 mM, 1-6 mM, 2-6 mM, or 3-6 mM when the IVT reaction is initiated. In some embodiments, the total NTP concentration in the IVT reaction is maintained above a lower limit of 0.5 mM. In some embodiments, the total NTP concentration in the IVT reaction is maintained at 10 mM to 20 mM.

In some embodiments, individual NTPs may be present at specific concentrations. For example, when each NTP is present at a concentration of 1-10 mM, each NTP may be present at a concentration of 4 mM. In some embodiments, each NTP is present at a concentration proportional to the number of occurrences of the NTP in the RNA transcript.

In some embodiments, a Raman spectrometer is used to monitor the amount of one or more NTPs in the reaction vessel. A Raman spectrometer has defined limits of detection (LoD) and limits of quantification (LoQ). The LoD and LoQ values for a Raman spectrometer are shown in Table 1. Accordingly, in some embodiments, the concentration at which the reaction vessels are supplemented with one or more NTPs is selected, taking into account the LoD and/or LoQ of the Raman spectrometer.

TABLE 1
Limits of detection and quantification of a Raman spectrometer

| | NTP (mM) | RNA (g/L) | HPO₄²⁻ (mM) |
|---|---|---|---|
| Limit of Detection | 1.7 | 0.6 | 1.0 |
| Limit of Quantification | 5.8 | 2.0 | 3.3 |

In some embodiments, a Raman spectrometer is used to monitor the amount of template and/or RNA polymerase during the IVT reaction. For example, changes in the amount of the template or the RNA polymerase may be indicative of contamination of the IVT reaction.

In some embodiments, a Raman spectrometer is used to monitor the addition of one or more enzymatic components such as a nuclease (e.g., DNase I) and/or a protease (e.g., proteinase K) to terminate the IVT reaction.

In some embodiments, a Raman spectrometer is used to monitor the amount of template to confirm destruction of this reagent during the termination phase of the IVT reaction.

In some embodiments, a Raman spectrometer is used to monitor the amount of RNA polymerase and/or nuclease (e.g., DNase I) to confirm destruction of these enzymes during the termination phase of the IVT reaction.

### Batch Size

The method of the invention can be used to optimize reaction conditions prior to the large-scale manufacturing of an RNA (e.g., an mRNA). Alternatively, the method of the invention may be used as a means of process and/or quality control during the large-scale manufacturing of RNA (e.g., mRNA).

Large-scale manufacturing typically involves the production of batches of RNA (e.g., mRNA) that are 100 mg or larger. Accordingly, in some embodiments, at least 100 mg of RNA is synthesized in a single batch. In some embodiments, at least 200 mg of RNA is synthesized in a single batch. In some embodiments, at least 300 mg of RNA is synthesized in a single batch. In some embodiments, at least 400 mg of RNA is synthesized in a single batch. In some embodiments, at least 500 mg of RNA is synthesized in a single batch. In some embodiments, at least 600 mg of RNA is synthesized in a single batch. In some embodiments, at least 700 mg of RNA is synthesized in a single batch. In some embodiments, at least 800 mg of RNA is synthesized in a single batch. In some embodiments, at least 900 mg of RNA is synthesized in a single batch.

In some embodiments, the term “large-scale manufacturing” refers to the production of batches of at least 1 g of RNA (e.g., mRNA). In some embodiments, at least 1 g of mRNA is synthesized in a single batch. In some embodiments, at least 5 g of RNA is synthesized in a single batch. In some embodiments, at least 10 g of RNA is synthesized in a single batch. In some embodiments, at least 25 g of RNA is synthesized in a single batch. In some embodiments, at least 50 g of RNA is synthesized in a single batch. In some embodiments, at least 75 g of RNA is synthesized in a single batch. In some embodiments, at least 100 g of RNA is synthesized in a single batch. In some embodiments, at least 150 g of RNA is synthesized in a single batch. In some embodiments, at least 200 g of RNA is synthesized in a single batch. In some embodiments, at least 250 g of RNA is synthesized in a single batch. In some embodiments, at least 500 g of RNA is synthesized in a single batch. In some embodiments, at least 750 g of RNA is synthesized in a single batch.

In some embodiments, the term “large-scale manufacturing” refers to the production of batches of at least 1 kg of RNA (e.g., mRNA). In some embodiments, at least 1 kg of RNA is synthesized in a single batch. In some embodiments, at least 5 kg of RNA is synthesized in a single batch. In some embodiments, at least 10 kg of RNA is synthesized in a single batch. In some embodiments, at least 10 kg of RNA is synthesized in a single batch. In some embodiments, at least 100 kg of RNA is synthesized in a single batch. In some embodiments, at least 1000 kg of RNA is synthesized in a single batch.

### Reaction Vessel

The IVT reaction is performed in a suitable reaction vessel, e.g., a bioreactor. In order to facilitate the monitoring of the IVT reaction, a spectroscopic probe is typically inserted into the reaction vessel. The use of a spectroscopic probe allows for the direct monitoring of the amounts of the one or more reactants and/or the one or more products during the course of the IVT reaction without disruption to the reaction e.g., for sample extraction. In some embodiments, the reaction vessel has a dedicated access port for insertion of a spectroscopic probe. In such embodiments, the spectroscopic probe is immersed directly in the solution in which the IVT reaction occurs. Separation of the spectroscopic probe and the solution directly can reduce the risk of contamination of the IVT reaction. Accordingly, in some embodiments, the spectroscopic probe is not directly immersed in the solution in which the IVT reaction occurs, e.g., the spectroscopic probe may be separated from the IVT reaction by a barrier that does not interfere with detection. In some embodiments, the spectroscopic probe is positioned in-line or in situ.

In some embodiments, it may be more convenient to incorporate the spectroscopic probe in a by-pass that branches off the reaction vessel. Placing the spectroscopic probe in a by-pass may be advantageous to avoid, e.g., background noise or other interference associated with agitation means provided within the reaction vessel.

In some embodiments, the reaction vessel additionally comprises an access port for the addition of reactants during the IVT reaction.

In some embodiments, the reaction vessel is configured for fed-batch or continuous operation.

Suitable reaction vessels may be made of glass, plastic or stainless steel. In some embodiments, the reaction vessel can be sterilised and sealed to avoid contamination (e.g., a single-use sterilisable and sealable plastic bag). In some embodiments, the reaction vessel may be heatable. In some embodiments, the reaction vessel is a bioreactor.

### Reaction Volume

The IVT reaction can be performed at a small scale, e.g., during optimization of an IVT reaction with a particular RNA of interest (e.g., an mRNA for therapeutic use). In some embodiments, the reaction vessel has a volume of at least 2 mL.

More typically, a reaction vessel (e.g., a bioreactor) is selected for optimization that allows to scale the IVT reaction to accommodate larger batches (e.g., for the commercial production of mRNA). In some embodiments, the reaction vessel has a volume of at least 200 mL. For large-scale manufacturing, a greater volume may be selected. In some embodiments, the reaction vessel has a volume of 12.5 L to 2000 L. In some embodiments, the reaction vessel has a volume of 500 L, 1000 L, or 2000 L or more.

### Means for Heating

Typically, the IVT reaction occurs at a temperature above room temperature, e.g., between 32° C. and 42° C. (e.g., 35° C.-39° C., or about 35° C., 36° C., 37° C., 38° C., or 39° C.). Accordingly, in some embodiments, the reaction vessel (e.g., a bioreactor) comprises a means for heating.

### Means for Agitating the IVT Reaction

In some embodiments, the IVT reaction occurs without agitation. In other embodiments, the IVT reaction is agitated, e.g., at 100 rpm to 400 rpm.

Accordingly, in some embodiments, the reaction vessel (e.g., a bioreactor) includes a means for providing agitation of the reaction mixture contained in it. For example, the reaction vessel may be programmable to provide agitation during the course of the IVT reaction. Accordingly, in some embodiments, the reaction vessel is configured to provide agitation. In some embodiments, agitation is at 100 rpm to 400 rpm.

### EXAMPLES

The following examples are included for illustrative purposes only and are not intended to limit the scope of the invention.

### Example 1. Quantitative Monitoring of RNA, NTP and Pi by Spectroscopy

This example illustrates the use of in-line spectroscopy for monitoring products and reactants during an IVT reaction.

IVT reactions were performed in the presence of NTPs and an RNA polymerase in a Tris reaction buffer (25 mM Tris pH7.5, 2 mM Spermidine, 25 mM MgCl₂, 5 mM NaCl), with 0.05 mg/mL linear template plasmid. The UTP was a modified UTP in which uridine was replaced with the uridine analogues such as N1-methylpseudouridine.

The coding region of the template encoded an exemplary ˜1.9 kb mRNA with a nucleotide sequence comprising 504 As, 490 Gs, 568 Cs, and 379 Us, respectively. Reactions were started by adding 1.1 ng/uL pyrophosphatase and 100 ng/uL RNA polymerase, and then incubated at 37° C. for 90 minutes. The IVT reactions were either conducted in a graduated cylinder in a total reaction volume of 2 mL without stirring or in a bioreactor system (Ambr250®, Sartorius) with a reaction volume of 250 mL with agitation at 250 rpm.

IVT reactions were analysed in-line (i.e., within the reaction vessel) using a ProCellics™ system (RESOLUTION Spectra Systems-MerckMillipore) for the acquisition of Raman spectra. This system uses a 785 nm excitation laser source of 350 mW power at the probe tip and a high-sensitivity spectrometer with 1 cm⁻¹ sampling step in the 150 cm⁻¹ to 4000 cm⁻¹ Raman shifts bandwidth (Stokes signal), using a back-thinned charge coupled device (CCD) detector. The spectroscopic probe was directly immersed into a 25 mL glass test tube filled with the 2 mL IVT reaction or the Ambr250® bioreactor filled at 250 mL. Aluminium foil layers were used to isolate the Raman measurement from any external light in order to guarantee the integrity of the analysis. An integration time of 50 seconds (averaging 10 spectra) was used for each acquisition. Pre-processing steps were performed with ProCellics™ Software (RESOLUTION Spectra Systems). Pre-processing included, as a first step, the generation of derivatives (order 1, at 15 cm⁻¹ steps, polynomial order 2) according to the Savitzky-Golay (SG) algorithm and, as a second step, the calculation of a customized Standard Normal Variate (SNV) between 3,100 cm⁻¹ and 3,600 cm⁻¹.

Empirical characteristic frequencies for chemical functional groups were used for spectral interpretation and compared to the molecular structure of IVT components. The peak at 810 cm⁻¹ was assigned to the phosphodiester bond (O—P—O symmetric stretching) and the peak at 1095 cm⁻¹ was assigned to PO₂⁻ symmetric stretching (O—P—O⁻), in agreement with published literature. The intensity ratio of these two peaks gives structural information about the RNA, namely the ratio between ordered and disordered structures. Since the phosphodiester bond is specific to RNA, the same characteristic frequency (810 cm⁻¹) can be monitored regardless of the polyribonucleotide sequence. During the IVT reaction, a new peak developed at 980 cm⁻¹ whose area seems to be correlated with the production of RNA. This peak was assigned to H₂PO₄⁻.

NTPs can be divided into 2 groups, those with a purine base (ATP and GTP) and those with a pyrimidine base (CTP and UTP). Adenine is a 6-monosubstituted purine. It is represented by a weak band that can be observed at 633 cm⁻¹ and can be attributed to the C—H out of plane bending vibration. Guanine is a 6-oxo-2-amino purine. CTP is represented as a band of medium intensity at 780 cm⁻¹ resulting from the cytosine ring vibrations. A band can also be observed at approximately 786-789 cm⁻¹ on the UTP spectrum, due to the stretching of the C═C bond (Carbon 5 and 6 of uracil). The peak around 1113-1115 cm⁻¹ decreased over the course of the IVT reaction and was attributed to NTP consumption. This peak could be associated with the PO₂⁻ stretching of the triphosphate group.

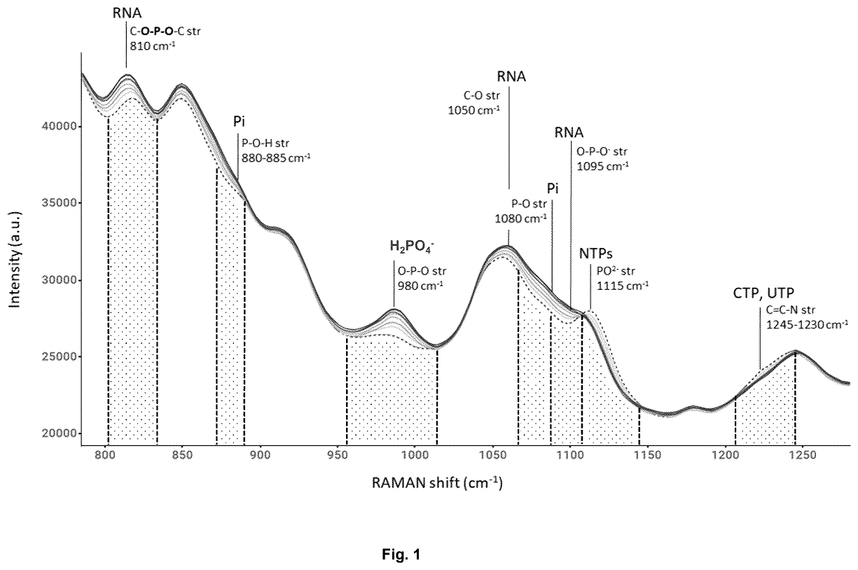

An illustrative Raman spectrum covering the region from 800 cm⁻¹ to 1300 cm⁻¹ is shown in FIG. 1. The dashed line represents the first recorded spectrum after 500 sec. from the beginning of the IVT reaction. The solid dark grey line is the last spectrum recorded of the IVT reaction after 90 min. As can be seen, regions of the spectrum corresponding to the RNA and the reaction by-product Pi show increased intensities after 90 minutes, whereas regions of the spectrum corresponding to NTPs show reduced intensities after 90 minutes.

This example demonstrates that in-line spectroscopy can be used to identify various components of an IVT reaction. “In-line” in this context means that the spectroscopic probe is immersed in the solution in which the IVT reaction occurs (e.g., in the reaction vessel or a by-pass of the reaction vessel). In-line spectroscopy can be used to monitor the production of the RNA and the formation of Pi as a by-product. It can also be used to monitor the consumption of NTPs during the IVT reaction. The methods described herein are not limited to Raman spectroscopy and can be adapted to identify suitable wavelength regions in a spectrum or a series of spectra that correspond to a reactant or product of interest using other spectroscopic methods, in particular light scattering-based methods.

### Example 2. Using Reference Spectra to Monitor Changes in Products and Reactants

This example illustrates that spectra obtained during an IVT reaction can be compared to pre-determined reference spectra of reactants and products of the IVT reaction to monitor changes in these reactants and products over time.

To monitor changes in reactants and products in the reaction vessel, spectra obtained during an IVT reaction were compared to a pre-determined spectrum of a reactant or product of interest using a percent weighted spectral differences (WSD) calculation. For the quantitation of spectral differences, the WSD calculation provides a weighting function based on relative signal magnitude. A WSD value close to zero indicates high similarity with the reference spectrum, while high WSD values are indicative of a large difference between the obtained spectrum and the predetermined spectrum. WSD values can be calculated using the following formula:

$$WSD = \sqrt{{\sum }_{i = 1}^{n} \frac{\vert {A}_{i} \vert}{n \cdot \overline{\vert A \vert}} {( {A}_{i} - {B}_{i} )}^{2}}$$

Aᵢ and Bᵢ are the intensity of spectrum A and B, respectively, at wavelength i.

Using Raman spectroscopy as described in Example 1, a 300 cm⁻¹ to 3000 cm⁻¹ wavelength region was selected for monitoring the global evolutions of spectra during an IVT reaction. For specific monitoring of RNA (product) and NTPs (reactants), a 801-831 cm⁻¹ wavelength region and a 1107-1146 cm⁻¹ wavelength region were selected, respectively.

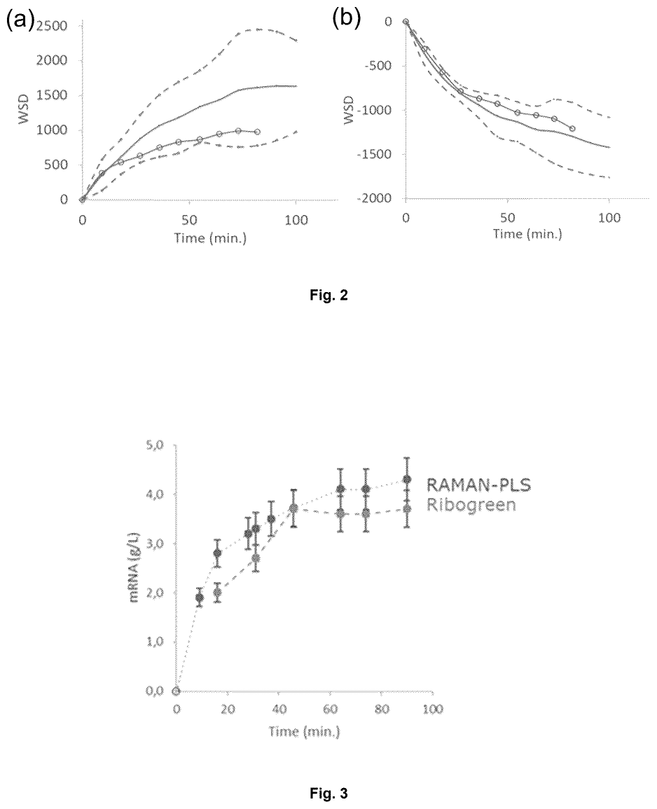

FIG. 2 illustrates the evolution of the WSD values for the 801-831 cm⁻¹ RNA region in panel (a) and the 1107-1146 cm⁻¹ NTP region in panel (b) during IVT reactions performed as described in Example 1. Both at the 2 mL scale and the 250 mL scale, the amount of RNA increased throughout the reaction reaching a plateau towards the end of the reaction time (FIG. 2, panel (a)). This was accompanied by a steady decrease in NTPs over the same time period (FIG. 2, panel (b)).

This example demonstrates that obtaining a series of spectra during an IVT reaction and comparing these spectra to pre-determined reference spectra of reactants and products of the IVT reaction can be used to determine changes in the concentration of the reactants and products over the course of the IVT reaction.

### Example 3. Constructions of Partial Least Square (PLS) Models

This example illustrates the construction of Partial Least Square (PLS) models using pre-determined spectra of reactants and products of IVT reactions at specified concentrations. The PLS models can be used as a prediction dataset to determine the respective concentrations of reactants and products from spectra obtained during an IVT reaction.

Like linear regression, a Partial Least Square (PLS) model aims to calculate a set of parameters connecting response variables (Y matrix) to independent variables (X matrix). PLS finds a linear regression model by projecting the response variables and the independent variables to a new space. The fundamental relationships are set between the variables in this new space and projected afterwards back into the original space. PLS regression is useful in predicting the response variables Y from a large set of independent variables X through reducing the set of X variables to a smaller set of uncorrelated components. The least squares regression is performed on these components, reducing thus the multi-co-linearity among the X values.

PLS is particularly useful for finding patterns in spectral signatures when relative chemical data are available. The SIMCA® software (version 16) was used to construct a PLS model on the Raman spectral data of 42 different mixtures comprising different concentrations of each of the NTPs, H₂PO₄⁻, and RNA in a buffer solution constructed as a D-optimal design using the JMP software (version 14). The D-optimal design was created by permutating the maximum and minimum concentrations of each component (see Table 2). The spectra of each of these 42 mixtures were recorded as described in Example 1.

TABLE 2
Concentrations of products and reactants

| Mixture | | | | | | |
|---|---|---|---|---|---|---|
| Number | [ATP] | [GTP] | [CTP] | [UTP] | [H₂PO₄⁻] | [RNA] |
| 1 | 5 | 5 | 5 | 5 | 15 | 0 |
| 2 | 5 | 0.4 | 0.4 | 5 | 15 | 0 |
| 3 | 0.4 | 5 | 5 | 0.4 | 15 | 0 |
| 4 | 0.4 | 0.4 | 0.4 | 5 | 0 | 0 |
| 5 | 5 | 5 | 0.4 | 5 | 0 | 0 |
| 6 | 0.4 | 5 | 0.4 | 5 | 15 | 0 |
| 7 | 0.4 | 5 | 0.4 | 0.4 | 0 | 0 |
| 8 | 0.4 | 5 | 5 | 5 | 0 | 0 |
| 9 | 5 | 5 | 5 | 0.4 | 0 | 0 |
| 10 | 5 | 0.4 | 0.4 | 0.4 | 0 | 0 |
| 11 | 0.4 | 0.4 | 0.4 | 5 | 0 | 0 |
| 12 | 5 | 5 | 5 | 0.4 | 0 | 0 |
| 13 | 0.4 | 5 | 5 | 0.4 | 15 | 0 |
| 14 | 5 | 5 | 5 | 5 | 15 | 0 |
| 15 | 5 | 0.4 | 5 | 0.4 | 15 | 0 |
| 16 | 5 | 0.4 | 5 | 0.4 | 15 | 0 |
| 17 | 0.4 | 0.4 | 0.4 | 0.4 | 15 | 0 |
| 18 | 5 | 0.4 | 0.4 | 0.4 | 0 | 0 |
| 19 | 0.4 | 0.4 | 5 | 5 | 15 | 0 |
| 20 | 0.4 | 0.4 | 5 | 0.4 | 0 | 0 |
| 21 | 5 | 5 | 0.4 | 5 | 0 | 0 |
| 22 | 0.4 | 5 | 0.4 | 0.4 | 0 | 0 |
| 23 | 2.7 | 2.7 | 2.7 | 2.7 | 7.5 | 0 |
| 24 | 5 | 0.4 | 0.4 | 5 | 15 | 0 |
| 25 | 0.4 | 5 | 0.4 | 5 | 15 | 0 |
| 26 | 5 | 0.4 | 5 | 5 | 0 | 0 |
| 27 | 5 | 0.4 | 5 | 5 | 0 | 0 |
| 28 | 2.7 | 2.7 | 2.7 | 2.7 | 7.5 | 0 |
| 29 | 5 | 5 | 0.4 | 0.4 | 15 | 0 |
| 30 | 0.4 | 5 | 5 | 5 | 0 | 0 |
| 31 | 0.4 | 0.4 | 5 | 0.4 | 0 | 0 |
| 32 | 2.7 | 2.7 | 2.7 | 2.7 | 7.5 | 0 |
| 33 | 0.4 | 5 | 0.4 | 5 | 15 | 0 |
| 34 | 6 | 0 | 0 | 0 | 0 | 0 |
| 35 | 0 | 4.6 | 0 | 0 | 0 | 0 |
| 36 | 0 | 0 | 5.5 | 0 | 0 | 0 |
| 37 | 0 | 0 | 0 | 3.9 | 0 | 0 |
| 38 | 0 | 0 | 0 | 0 | 15 | 0 |
| 39 | 3 | 2.3 | 0 | 0 | 0 | 0 |
| 40 | 0 | 0 | 2.25 | 1.95 | 0 | 0 |
| 41 | 6 | 4.6 | 5.5 | 3.9 | 0 | 0 |
| 42 | — | — | — | — | — | 4 |

PLS models were constructed for each of the parameters ATP-GTP, CTP-UTP, Pi, and RNA based on the spectra of the 42 mixtures. Raman signatures of the adenine and guanine bases overlapped and were not distinguishable in the PLS models due to the high correlation of these parameters. The same was observed for the cytosine and uracil bases. In consequence, ATP and GTP as well as CTP and UTP were quantified by summing their concentrations and the two identified parameters were ATP-GTP and CTP-UTP, respectively. The PLS models were then used as a prediction dataset to calculate the respective concentrations of the reactants and the products in respect to time. The PLS SIMCA file was imported to the ProCellics software of the Raman spectrometer to monitor the concentrations of the reactants and products in the reaction vessel during IVT reactions, as illustrated in Example 4.

### Example 4. Monitoring RNA Production in the Reaction Vessel

This example illustrates that spectroscopy can be used to determine the RNA concentration within a reaction vessel during an IVT reaction.

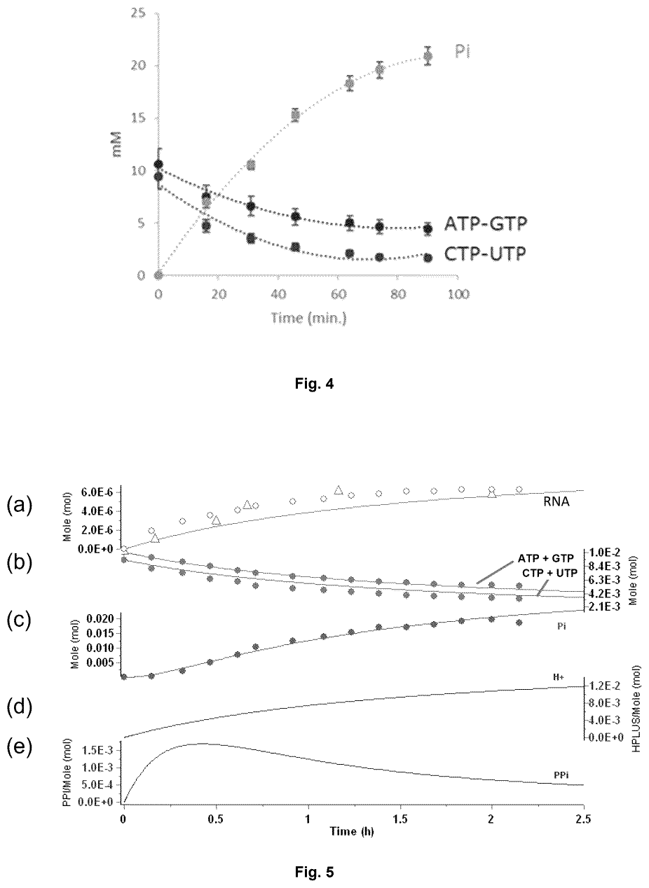

The concentration of RNA is zero at the beginning of the IVT reaction (t=0 minutes) and increases over time after transcription is initiated with the addition of the RNA polymerase as described in Example 1. Using in-line Raman spectroscopy and the PLS model described in Example 3, the RNA concentration at 18.3 minutes was determined to be 2.8 g/L. The concentration increased to 4.1 g/L at 64.1 minutes. These values were compared to the results of an off-line quantification using RiboGreen assay. Aliquots were removed from the IVT reaction at various time points. The concentrations calculated from the RiboGreen assay data were 2.0 g/L at 16 minutes and 3.5 g/L at 62 minutes. FIG. 3 plots the concentration values obtained with Raman spectroscopy relative to the concentration values obtained with the RiboGreen assay.

For the RiboGreen assay, the RiboGreen® reagent (ThermoFisher) was diluted 200-fold in TE buffer, and the reagent solution was added to an equal volume of RNA in TE. Samples in microplate were incubated for 5-30 min at room temperature, protected from light. Sample volumes for microplate assays were 200 mL. Microplate assays were performed using a CytoFluor II fluorescence microplate reader (Soft Max Pro 6.5.1). Samples were excited at 495 nm, and fluorescence was measured at 521 nm. Integrated fluorescence emission intensities were plotted versus RNA concentration, with no subtraction of background fluorescence. The relative error of the RiboGreen assay was 12% while the relative error of the PLS model (described in Example 3) was 11%.

This example demonstrates that spectroscopy can be used to determine the RNA concentration during an IVT reaction within a reaction vessel. The in-line spectroscopy method yielded concentrations that are comparable to standard off-line methods such as the RiboGreen assay.

### Example 5. Monitoring NTP Consumption and Pi Production in the Reaction Vessel

This example illustrates that spectroscopy can be used to monitor NTP consumption and the accumulation of inorganic phosphate (Pi) during an IVT reaction within a reaction vessel.

Magnesium (Mg²⁺) is an essential cofactor of RNA polymerases and has a direct impact on the rate of the transcription, and, as a result, on the production of the RNA. It can combine with the pyrophosphate (PPi) and lead to the formation of an insoluble precipitate that can affect the RNA yield during production. Pyrophosphatase is added to the IVT reaction to hydrolyse PPi and enhance the RNA yields. This results in the production of inorganic phosphate (Pi). The concentration of Pi is zero at the beginning of the IVT reaction (t=0 minutes) and increases over time after transcription is initiated with the addition of the RNA polymerase as described in Example 1. As ribonucleotides (NTPs) are incorporated into RNA, their concentration within the reaction vessel diminishes.

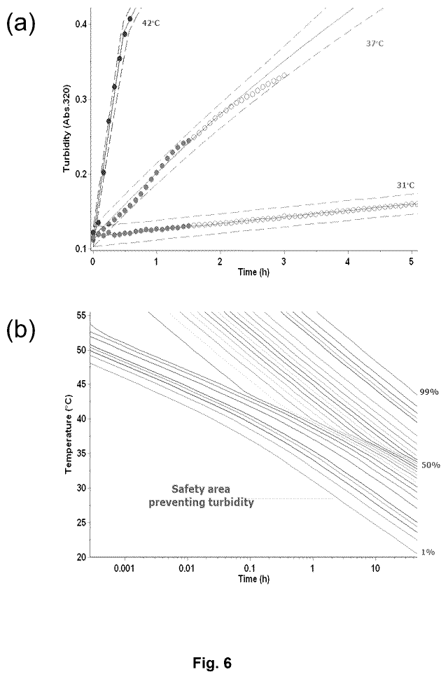

Using in-line Raman spectroscopy and the PLS model described in Example 3, the concentrations of ATP-GTP, CTP-UTP, and Pi were monitored within the reaction vessel. As expected, intensity of the Raman spectra in the selected spectral regions decreased and increased, respectively, indicating a decrease in the concentrations of ATP-GTP and CTP-UTP and an increase of the Pi concentration during the IVT reaction. Representative spectra are shown in FIG. 4. The relative error of the calculated values is 14% and 13% for the ATP-GTP and CTP-UTP concentrations, respectively, and 4% for the Pi concentration.

This example demonstrates that spectroscopy can be used to monitor NTP and pyrophosphate concentrations during an IVT reaction within a reaction vessel. The in-line information provided by spectroscopy can be valuable in determining whether the NTP concentration may require adjustment.

### Example 6. Building a Kinetic Model for IVT Reactions

This example illustrates how a kinetic model of the IVT reaction described in Example 1 is consistent with experimental observations made using inter alia the spectroscopic method described herein.

The reaction stoichiometry for net synthesis of an RNA transcript consisting of n nucleotides is given by the following equation:

$$nA ATP + nG GTP + nC CTP + nU UTP + DNA \to RNAn + ( n - 1 ) PPi + DNA$$

nA, nG, nC, and nU represent the number of adenine, guanine, cytosine, and uracil bases, respectively, in each copy of the fully transcribed RNA, in which the sum of nucleotides equals n (=nA+nG+nC+nU). Using the RNA encoded by the template plasmid described in Example 1, the following mechanistic scheme was adopted for reaction 1:

$$504 ATP + 490 GTP + 568 CTP + 379 UTP \to 1 RNA + 1940 PPi + 1940 {H}^{+}$$

H⁺ can either be expressed solely, as being in solution, or considered as bound to the PPi. The presence of pyrophosphate (PPi) was shown to inhibit the RNA synthesis reaction. This is why a second reaction is needed in order to hydrolyse to PPi into Pi with the help of a pyrophosphatase. The following mechanistic scheme was adopted for reaction 2:

$$1940 PPi + 1940 {H}_{2} O \to 3880 Pi$$

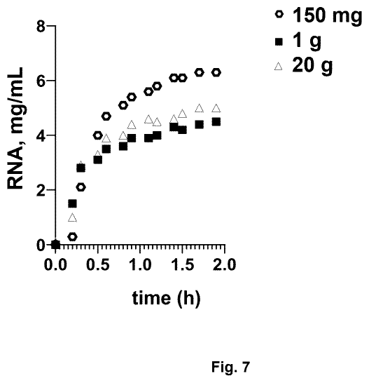

By using concentrations of NTPs and Pi determined using a PLS model as described in Example 3 and assuming that reactions 1 and 2 occur during the IVT reaction, the best fit of data led to predictions of concentration of RNA and PPi as illustrated in FIG. 5. Based on this analysis, a kinetic model could be formulated as follows:

$$r 1 = ( 4.795 \times {10}^{- 4} ) \times {[ ATP + GTP ]}^{1} \times {[ CTP + UTP ]}^{1}$$

$$and$$

$$r 2 = ( 1.805 \times {10}^{- 5} ) \times {[ PPi ]}^{1} \times {[ {H}_{2} O ]}^{1}$$

4.795×10⁻⁴+ and 1.805×10⁻⁵ are rate constants in g/(s×mol) for r1 and r2, respectively.

Using this empirical model, ATP-GTP, CTP-UTP, Pi, and RNA concentrations were predicted. The RNA kinetic profile was compared to the set of concentrations calculated by means of the PLS model (represented in open circles) and to the RiboGreen quantification values (represented by open triangles) described in Example 4 (see panel (a) of FIG. 5). Agreement between predictions (kinetic model) and experimental data (Raman-PLS and RiboGreen) was observed, confirming the ability of spectroscopy combined with the PLS method described in Example 3 to quantify in-line RNA production.

Beyond the estimation of RNA content, the kinetic model estimated the progress of other reactants and products, including PPi (see panel (e) of FIG. 5), showing first an increase of PPi, then a slow decrease. This behaviour seems to align with reaction 1 (production of PPi) and reaction 2 (degradation of PPi by pyrophosphatase). Furthermore, the modelled increase of H′ (see panel (d) of FIG. 5) was in agreement with a decrease in pH during the IVT reaction, as previously observed.

### Example 7. Monitoring and Modelling the Emergence of Turbidity

This example illustrates that spectroscopy can be used to monitor the emergence of turbidity during an IVT reaction which results from the build-up of insoluble components such as Mg₂PPi precipitates as reaction by-products.

Emergence of turbidity was monitored during IVT reactions at three different temperatures (31° C., 37° C., 42° C.). Absorbance measurements were performed during a run time of 90 minutes, with an interval of 2 minutes between measurements. The turbidity was measured with a SpectraMax M5 UV-visible spectrophotometer (Molecular DevicesR). The presence of insoluble components such as Mg₂PPi precipitates was estimated by measuring the absorbance at 320 nm.

A kinetic model was developed combining advanced kinetics and statistical analysis of stability data obtained at 31° C., 37° C. and 42° C. The rate at which turbidity emerged was temperature-dependent, enabling an Arrhenius-based modelling. Turbidity emerged more rapidly the higher the temperature at which the IVT reaction was performed. Advanced kinetic modelling by fitting of the collected data was applied, leading to a two-step kinetic model to describe the reaction progress as function of time and temperature.

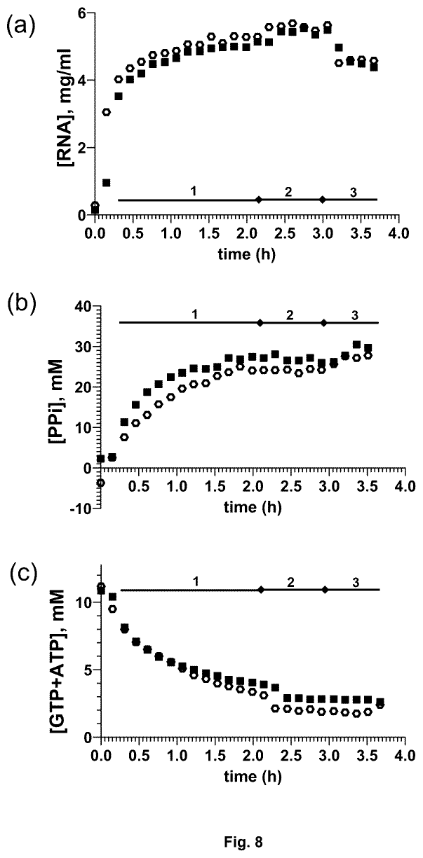

This model was used for predictions of longer-term turbidity levels, up to 5 hours (see panel (a) of FIG. 6). It was also employed to estimate a safety area delimiting a time-temperature domain in which the emergence of turbidity can be prevented (see panel (b) of FIG. 6).

This example demonstrates that the spectroscopic measurement of turbidity during an IVT reaction can be used as a method of monitoring the formation of reaction by-products such as Mg₂PPi precipitates.

### Example 8. Scalability

This example illustrates that the progress of an IVT reaction can be monitored reproducibly with the methods disclosed in the preceding examples as the batch size increases or as the IVT reaction is performed using a fed-batch system.

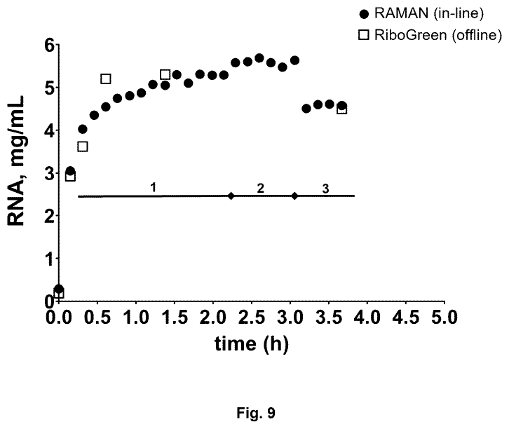

Quantitative monitoring of RNA during an IVT reaction using in-line Raman spectroscopy and the PLS model described in Example 3 was performed. The IVT reaction was performed as described in Example 1 for a duration of 2 hours but using a different DNA template encoding a ˜2 kb mRNA with a nucleotide sequence comprising 609 As, 449 Cs, 522 Gs, and 392 Us. The progress of the IVT reaction, as measured by the increase in concentration of RNA, was comparable irrespective of the RNA batch size (150 mg, 1 g, or 20 g; see FIG. 7).

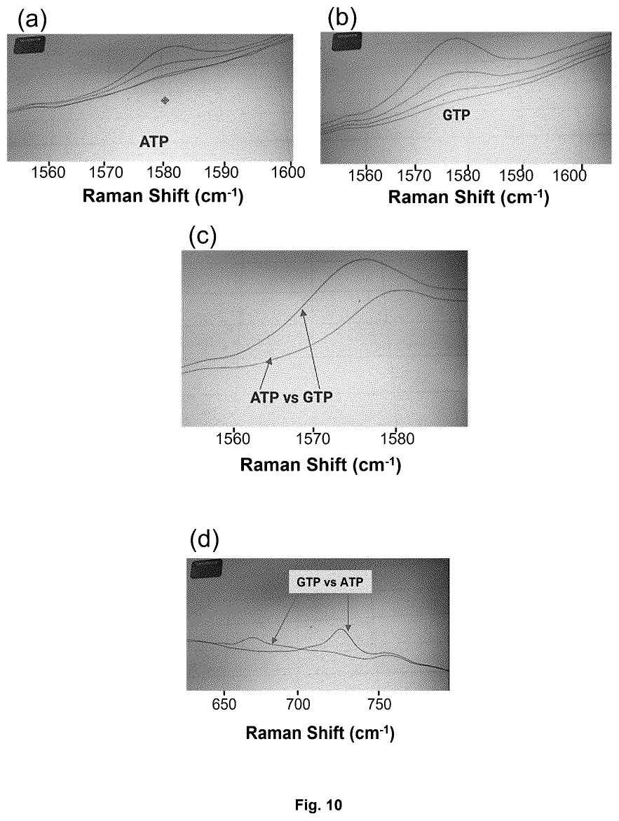

The scalability of the monitoring methods disclosed herein was also observed with another DNA template encoding a ˜2 kb mRNA with a nucleotide sequence comprising 554 As, 492 Cs, 517 Gs, and 406 Us (see FIG. 8). Comparable results between two different batch sizes (1 g and 20 g) were obtained when monitoring the products RNA and PPi, and the nucleotide reactants, as illustrated by the amount of GTP-ATP. For the 20 g batch, the IVT reaction was performed in a 5000 L reaction vessel.

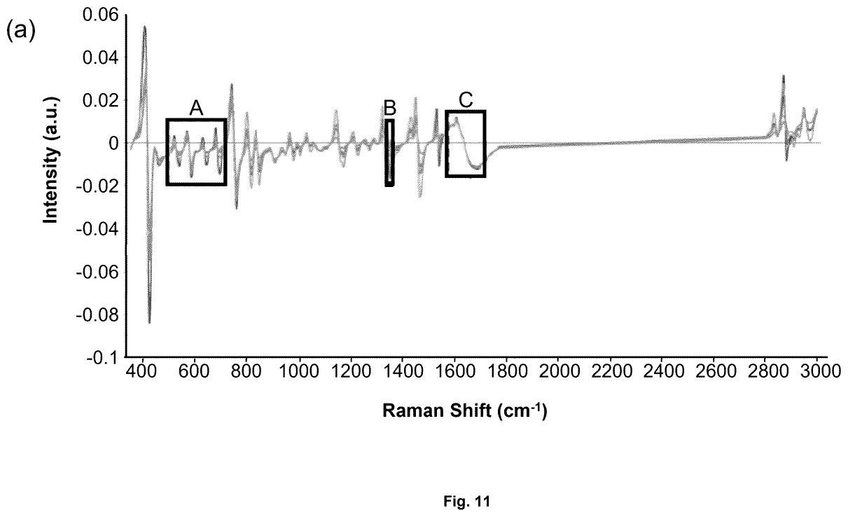

Consistent with Example 4, the RNA concentration determined from in-line quantification (Raman) and off-line quantification (RiboGreen assay) were comparable. As shown in FIG. 9, mRNA is shown to increase as the IVT reaction progresses which ceases upon addition of DNase I (to digest the template). Following template digestion, proteinase K was added to digest the enzymatic components including the RNA polymerase as well as the DNase I. A drop in the amount of mRNA was observed following the addition of DTT to quench the reaction. This was due to dilution of the sample by the addition of DTT rather than loss of the mRNA product.

In addition, the applicability of the modelling system described in Example 3 was tested in IVT reactions with four different DNA templates. The obtained spectra could be fitted within the previously determined mathematical model parameters for predicting the progression of the IVT reaction.

In addition, an IVT reaction was also performed in a reaction vessel using fed-batch operation. The monitoring methods described in the preceding examples worked equally well under these conditions.

This example demonstrates that the monitoring methods described in the preceding examples can be used at the batch size is increased or as IVT reaction is performed using a fed-batch system.

### Example 9. Monitoring Individual NTPs

This example illustrates that spectroscopy can be used to monitor individual NTPs.

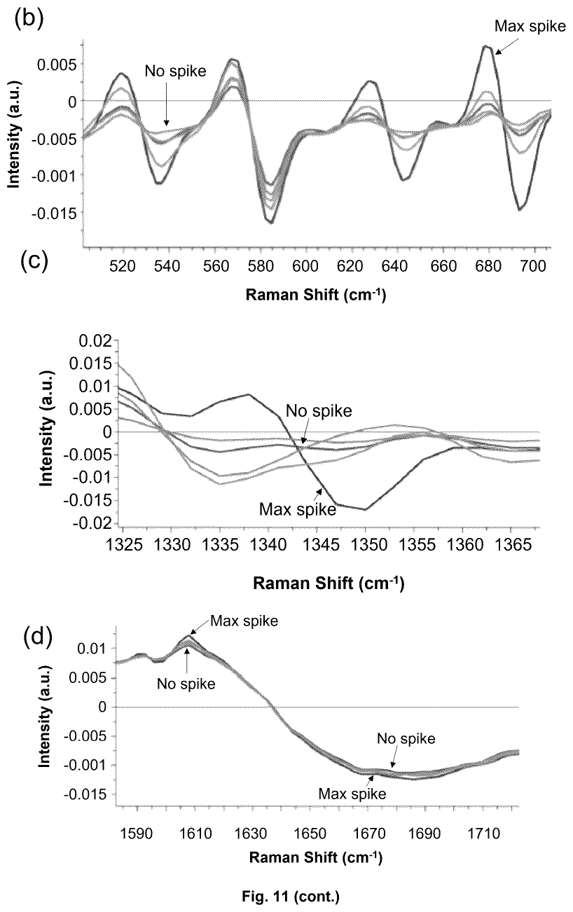

Various concentrations of ATP and GTP were tested by spiking in a reaction buffer to identify reactant zones. Raman spectra were acquired using a Kaiser Raman R×n2 analyzer (Endress+Hauser). Using this system, ATP and GTP could be identified in the wavelength regions of 1550-1600 cm⁻¹ and 650-750 cm⁻¹, respectively, as illustrated in FIG. 10. Overlaying spectra obtained for ATP and GTP in the region of 650-750 cm⁻¹ showed discrete regions corresponding to each nucleotide, as illustrated in FIG. 10.

### Example 10. Monitoring Reaction Termination in the Reaction Vessel

This example demonstrates that the spectroscopic methods described in the preceding examples can also be used to monitor the termination of an IVT reaction.

The feasibility of using spectrometry to monitor the termination of an IVT reaction was investigated. Various concentrations of template (a DNA plasmid), nuclease (DNase I), or RNA polymerase (SP6 RNA polymerase) were spiked into the reaction buffer to identify suitable wavelengths for monitoring these components.

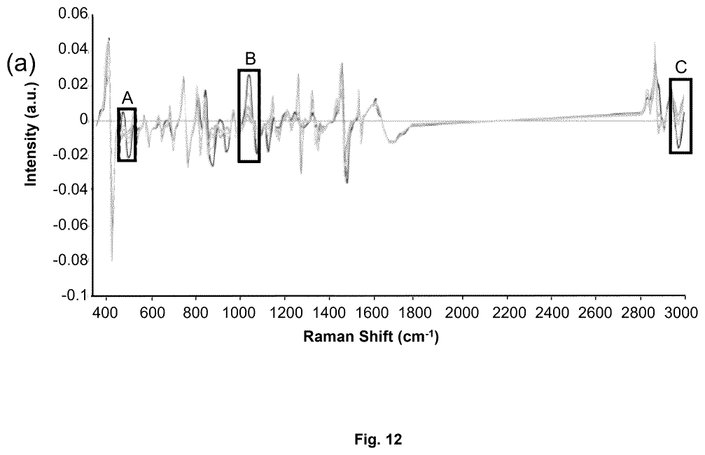

FIG. 11 shows illustrative spectra obtained when testing DNA plasmid at concentrations of 0 mg/mL, 0.075 mg/mL, 0.15 mg/mL, 0.3 mg/mL, and 0.6 mg/mL. The reaction buffer did not include RNA polymerase to avoid the progression of the IVT reaction. A suitable wavelength region to monitor the DNA plasmid was identified as 505 cm⁻¹ to 710 cm⁻¹. Other suitable wavelength regions for monitoring the amount of DNA plasmid included 1325 cm⁻¹ to 1365 cm⁻¹ and 1585 cm⁻¹ to 1720 cm⁻¹. Monitoring changes in DNA plasmid concentrations can be used to monitor the termination of the IVT reaction termination e.g., by the addition of a nuclease such as DNase I.

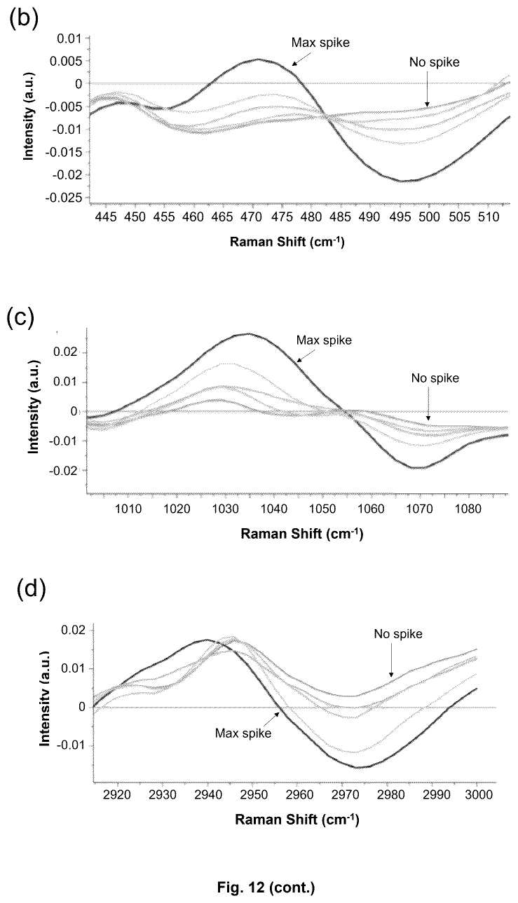

FIG. 12 shows illustrative spectra obtained when testing DNase I at concentrations of 0 ku/mL, 0.063 ku/mL, 0.125 ku/mL, 0.25 ku/mL, and 0.5 ku/mL. The reaction buffer did not include the DNA template. A suitable wavelength region to monitor a nuclease such as DNase I was identified as comprising 450 cm⁻¹ to 520 cm⁻¹. Other suitable wavelength regions for monitoring the amount of the nuclease included 1000 cm⁻¹ to 1090 cm⁻¹ and 2915 cm⁻¹ to 3000 cm⁻¹. The spectra obtained from monitoring DNase I can be used to determine whether it was added but also to detect its removal e.g., by digestion with a protease such as proteinase K.

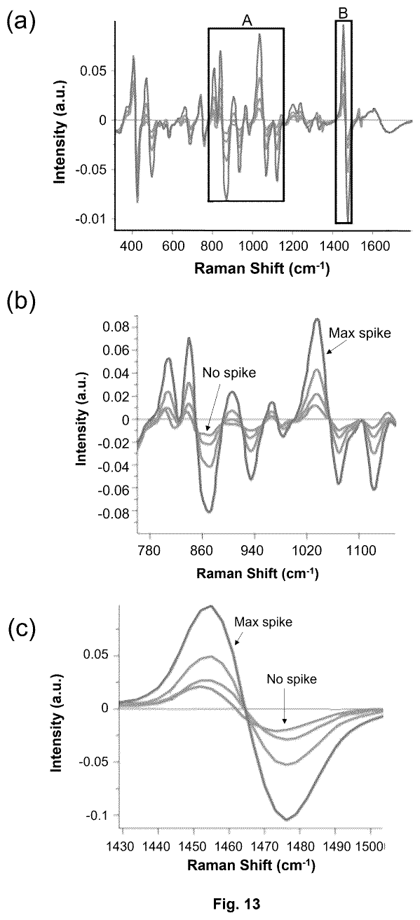

FIG. 13 shows illustrative spectra obtained when testing SP6 RNA polymerase at concentrations of 0 mg/mL, 0.045 mg/mL, 0.09 mg/mL, 0.18 mg/mL, and 0.36 mg/mL. The reaction buffer did not include a DNA template to avoid the progression of the IVT reaction. A suitable wavelength region to monitor SP6 RNA polymerase was identified as 780 cm⁻¹ to 1200 cm⁻¹. Another suitable wavelength region for monitoring the amount of the RNA polymerase included 1430 cm⁻¹ to 1510 cm⁻¹. Monitoring changes in SP6 RNA polymerase allows the user to detect its removal during termination of the IVT reaction, e.g., by digestion with a protease such as proteinase K.

Similar spiking experiments were also performed to identify a wavelength region to detect proteinase K, the protease which is added to digest the RNA polymerase to terminate the IVT reaction or to digest DNase I and terminate the DNase I reaction. In some embodiments, the spectra obtained in step (c) are used to monitor the amount or addition of the protease. A suitable wavelength region to monitor proteinase K was identified as comprising 505 cm⁻¹ to 610 cm⁻¹, e.g., 550 cm⁻¹ to 600 cm⁻¹. Other suitable wavelength regions for monitoring the amount of the protease included 715 cm⁻¹ to 775 cm⁻¹ and to 1385 cm⁻¹ to 1395 cm⁻¹.

This example demonstrates that the spectroscopic methods used for monitoring an IVT reaction can also be used to monitor the removal of DNA plasmid and enzymatic components that are employed to terminate the reaction.

1. A method for monitoring an in vitro transcription (IVT) reaction for the production of RNA within a reaction vessel, said method comprising:
(i) obtaining a spectrum of a reactant or product during the IVT reaction; and
(ii) comparing the spectrum obtained in step (i) to a pre-determined reference spectrum of the reactant or product of the IVT reaction;
wherein a difference between the spectrum obtained in step (i) and the pre-determined reference spectrum indicates a change in the amount of the reactant or product.

2. The method of claim 1, wherein the reactant or product is selected from RNA, pyrophosphate (PPi), H⁺, inorganic phosphate (Pi), and a ribonucleotide (NTP).

3. The method of claim 2, wherein the product is RNA or Pi, optionally wherein the RNA is an mRNA.

4. The method of claim 2, wherein the reactant is one or more ribonucleotides (NTPs).

5. The method of any one of the preceding claims, wherein the method determines the change in the amount of more than one reactant or more than one product of the IVT reaction.

6. The method of claim 5, wherein the method determines the change in the amount of more than one reactant and more than one product of the IVT reaction.

7. The method of claim 5 or 6, wherein the more than one reactants are adenosine triphosphate (ATP) and guanosine triphosphate (GTP).

8. The method of claim 5 or 6, wherein the more than one reactants are cytidine triphosphate (CTP) and uridine triphosphate (UTP).

9. The method of any one of claims 5-8, wherein the more than one products are RNA and Pi.

10. The method of any one of the preceding claims, wherein the reaction vessel is a bioreactor.

11. The method of any one of the preceding claims, wherein the reaction vessel has an access port or a by-pass for insertion of a spectroscopic probe.

12. The method of claim 11, wherein the spectroscopic probe is (a) immersed in the solution in which the IVT reaction occurs; or (b) not immersed in the solution in which the IVT reaction occurs, optionally wherein the spectroscopic probe is separated from the solution by a barrier that does not interfere with detection.

13. The method of any one of the preceding claims, wherein step (i) comprises obtaining a series of spectra over the course of the IVT reaction.

14. The method of claim 13, wherein each spectrum in the series is obtained over a time period of 10-60 seconds.

15. The method of claim 13 or 14, wherein the series of spectra comprises a set of at least 3, at least 5, or at least 9 spectra.

16. The method of any one of claims 13-15, wherein multiple series of spectra are obtained over the course of the IVT reaction.

17. The method of any one of claims 13-16, wherein each of the spectra in the series is pre-processed prior to step (ii).

18. The method of claim 17, wherein the spectra in the series are acquired successively and pre-processing comprises smoothing the spectra by applying a digital filter that fits the successively acquired spectra with a low-degree polynomial by a method of linear least squares.

19. The method of any one of the preceding claims, wherein the spectrum or series of spectra obtained in step (i) is/are normalised relative to a wavelength region with reduced or no background noise prior to step (ii).

20. The method of claim 17 or 18, wherein the series of spectra are normalised relative to a wavelength region with reduced or no background noise prior to any pre-processing step.

21. The method of claim 17, wherein step (ii) comprises a qualitative spectral comparison between the series of spectra obtained in step (i) and the pre-determined reference spectrum.

22. The method of claim 21, wherein the qualitative spectral comparison comprises calculating a weighted spectral difference (WSD) value.

23. The method of claim 22, wherein a WSD value of one standard deviation or less indicates that there is no significant difference between the spectrum obtained in step (i) and the pre-determined reference spectrum.

24. The method of claim 22, wherein a WSD value of more than one standard deviation indicates that there is a significant difference between a spectrum obtained in step (i) and the pre-determined reference spectrum.

25. The method of any one of the preceding claims, wherein the pre-determined reference spectrum correlates to a specified concentration of the reactant or product.

26. The method of claim 25, wherein step (ii) further comprises determining the concentration of the reactant or product.

27. The method of claim 26, wherein determining the concentration of the reactant or product comprises linear regression analysis.

28. The method of claim 26 or 27, wherein a partial least square (PLS) model is used to determine the concentration of the reactant or product from a series of pre-determined reference spectra, wherein each spectrum of the series correlates to different concentrations of the reactant or product.

29. The method of any one of the preceding claims, wherein the spectrum, series of spectra and the pre-determined reference spectrum/series of pre-determined reference spectra, as applicable, are obtained using a spectrometer for vibrational spectroscopy.

30. The method of claim 29, wherein the spectrometer is a Raman spectrometer.

31. The method of claim 29, wherein the spectrometer is an infrared (IR) spectrometer.

32. The method of any one of the preceding claims, wherein the spectrum obtained in step (i) spans a wavelength region suitable for monitoring the global evolution of multiple reactants and products during the IVT reaction.

33. The method of claim 32, wherein the multiple reactants and products include RNA, inorganic phosphate (Pi) and one or more ribonucleotides (NTPs).

34. The method of claim 32 or 33, wherein the wavelength region includes 300 cm⁻¹ to 3000 cm⁻¹ and the spectrum is obtained using Raman spectrometer.

35. The method of any one of claims 1-31, wherein the spectrum obtained in step (i) spans a wavelength region specific to one product or reactant of the IVT reaction.

36. The method of claim 35, wherein the product is RNA, optionally wherein the product is mRNA.

37. The method of claim 36, wherein the wavelength region includes 801 cm⁻¹ to 831 cm⁻¹ and the spectrum is obtained using a Raman spectrometer.

38. The method of claim 35, wherein the product is Pi.

39. The method of claim 38, wherein the wavelength region includes 875 cm⁻¹ to 900 cm⁻¹ and the spectrum is obtained using a Raman spectrometer.

40. The method of claim 35, wherein the reactant is one or more ribonucleotides (NTPs).

41. The method of claim 40, wherein the wavelength region comprises 600 cm⁻¹ to 1,300 cm⁻¹ and the spectrum is obtained using a Raman spectrometer.

42. The method of claim 40 or 41, wherein the wavelength region comprises or consists of 1107 cm⁻¹ to 1146 cm⁻¹ or 1113 cm⁻¹ to 1115 cm⁻¹ to determine the amount of NTPs.

43. The method of any one of claims 40-42, wherein the wavelength region comprises or consists of 633 cm⁻¹ to determine the amount of ATP.

44. The method of any one of claims 40-42, wherein the wavelength region comprises or consists of 1300 cm⁻¹ to 1600 cm⁻¹ to determine the amount of ATP and GTP.

45. The method of any one of claim 40-42, wherein the wavelength region comprises or consists of 780 cm⁻¹ to determine the amount of CTP.

46. The method of any one of claims 40-42, wherein the wavelength region comprises or consists of 786 cm⁻¹ to 789 cm⁻¹ to determine the amount of UTP.

47. The method of any one of claims 40-42, wherein the wavelength region comprises or consists of 1230 cm⁻¹ to 1245 cm⁻¹ to determine the amount of CTP and UTP.

48. The method of any one of claims 1-28, wherein the spectrum obtained in step (i) detects the turbidity of the solution in which the IVT reaction occurs.

49. The method of claim 48, wherein the turbidity indicates the accumulation of an insoluble precipitate.

50. The method of claim 49, wherein the insoluble precipitate is Mg₂PPi.

51. The method of claim 48-50, wherein turbidity is measured using a turbidimeter, a UV spectrometer, or a nephelometer.

52. The method of claim 51, wherein the turbidity is measured at a wavelength in the region of 290 nm to 410 nm, e.g., at 310 nm, 320 nm, 330 nm, 340 nm or 350 nm using a UV spectrometer.

53. A method of manufacturing an RNA using an in vitro transcription (IVT) reaction, said method comprising:
a) providing a DNA template comprising the nucleotide sequence of the RNA operationally linked to an RNA polymerase promoter;
b) adding the DNA template to a reaction vessel comprising an RNA polymerase and necessary reactants to initiate the IVT reaction; and
c) monitoring one or more of the reactants or products of the IVT reaction using the method of any one of the preceding claims.

54. The method of claim 53, wherein step (c) further comprises obtaining a series of spectra spanning a wavelength region suitable for monitoring the DNA template and/or one or more enzymatic component(s) such as the RNA polymerase during the IVT reaction and, optionally, after completion of the IVT reaction.

55. The method of claim 53 or 54, wherein the one or more products comprises RNA, optionally wherein the one or more products comprises mRNA.

56. The method of claim 55, wherein step (i) comprises obtaining a series of spectra at one or more specified time points during the IVT reaction and step (ii) comprises determining the amount, or change in the amount, of RNA between the one or more specified time points by comparing the series of spectra and the pre-determined reference spectrum.

57. The method of claim 56, wherein the IVT reaction is terminated if the RNA does not reach a target amount at the one or more specified time points.

58. The method of claim 57, wherein the batch of RNA resulting from the IVT reaction is discarded.

59. The method of claim 56, wherein the IVT reaction is terminated if the change in the amount of RNA is less than a pre-determined value at two or more specified time points.

60. The method of claim 59, wherein the batch of RNA resulting from the IVT reaction is discarded.

61. The method of claim 59 or 60, wherein the two or more specified time points are at equally-spaced intervals throughout the IVT reaction.

62. The method of claim 61, wherein each interval is 10 minutes or less, 5 minutes or less, 1 minute or less, or 30 seconds or less.

63. The method of claim 56, wherein the IVT reaction is terminated if the change in the amount of RNA is about zero between at least two or more time points.

64. The method of claim 63, wherein the at least two or more time points are at least 5 minutes apart.

65. The method of claim 53 or 54, wherein the IVT reaction is terminated if the spectrum of the IVT reaction obtained in step (i) diverges by more than one standard deviation from (A) the pre-determined reference spectrum of the reactant or product, or (B) a kinetic model previously determined for an IVT reaction using similar or identical conditions and reactants.

66. The method of claim 53 or 54, wherein the batch of RNA resulting from the IVT reaction is discarded if the spectrum of the IVT reaction obtained in step (i) diverges by more than one standard deviation from (A) the pre-determined reference spectrum of the reactant or product, or (B) a kinetic model previously determined for an IVT reaction using similar or identical conditions and reactants.

67. The method of any one of claims 54-66, wherein the spectra obtained in step (c) are used to monitor the amount of DNA template.

68. The method of claim 67, wherein the wavelength region obtained in step (c) comprises or consists of 500 cm⁻¹ to 710 cm⁻¹ to determine the amount of DNA template.

69. The method of any one of claims 54-68, wherein the spectra obtained in step (c) are used to monitor the amount of RNA polymerase, optionally wherein the RNA polymerase is SP6 RNA polymerase.

70. The method of claim 69, wherein the wavelength region obtained in step (c) comprises or consists of 780 cm⁻¹ to 1200 cm⁻¹ or 1430 cm⁻¹ to 1510 cm⁻¹ to determine the amount of RNA polymerase.

71. The method of any one of claims 54-70, wherein the method comprises adding a nuclease (e.g., DNase I) to terminate the IVT reaction.

72. The method of claim 71, wherein the spectra obtained in step (c) are used to monitor the amount or addition of the nuclease.

73. The method of claim 72, wherein the wavelength region obtained in step (c) comprises or consists of 450 cm⁻¹ to 520 cm⁻¹ to determine the amount or addition of the nuclease.

74. The method of any one of claims 54-73, wherein the method comprises adding a protease (e.g., proteinase K) to terminate the IVT reaction or nuclease activity.

75. The method of claim 74, wherein the spectra obtained in step (c) are used to monitor the amount or addition of the protease.

76. The method of claim 75, wherein the wavelength region obtained in step (c) comprises or consists of 505 cm⁻¹ to 610 cm⁻¹, optionally 550 cm⁻¹ to 600 cm⁻¹, to determine the amount or addition of the protease.

77. A method of manufacturing an RNA using an in vitro transcription (IVT) reaction, said method comprising:
a) monitoring the production of the RNA within a reaction vessel by:
(i) obtaining a spectrum of the RNA during the IVT reaction to determine a first value; and
(ii) comparing the first value obtained in step (i) to a second value derived from a pre-determined reference spectrum of RNA; and
b) purifying the RNA if the first value is equal to, or exceeds, the second value.

78. The method of claim 77, wherein the second value corresponds to a target concentration.

79. The method of claim 78, wherein the target concentration is at least 3 g/L.

80. The method of claim 77, wherein steps (i) and (ii) are repeated at equally spaced intervals and the RNA is purified if the first value obtained at each interval is equal to or exceeds the second value at corresponding intervals derived from the pre-determined reference spectrum.

81. The method of any one of claims 77-80, wherein the first and second values are considered equal if they are within one standard deviation of each other.

82. The method of any one of claims 53-81, wherein the reaction vessel is a bioreactor.

83. The method of any one of claims 53-82, wherein the reaction vessel has an access port or a by-pass for insertion of a spectroscopic probe.

84. The method of claim 83, wherein the spectroscopic probe is (a) immersed in the solution in which the IVT reaction occurs; or (b) not immersed in the solution in which the IVT reaction occurs, optionally wherein the spectroscopic probe is separated from the solution by a barrier that does not interfere with detection.

85. The method of any one of claims 53-84, wherein the reaction vessel has an access port for the addition of reactants during the IVT reaction.

86. The method of any one of claims 53-85, wherein the reactants in the IVT reaction include magnesium (Mg²⁺) and NTPs.

87. The method of claim 86, wherein the IVT reaction is supplemented at least once with NTPs during the course of manufacturing the RNA.

88. The method of claim 87, wherein the IVT reaction is supplemented periodically with NTPs during the course of manufacturing the RNA.

89. The method of claim 87 or 88, wherein the IVT reaction is supplemented with NTPs when the concentration of NTPs approaches depletion.

90. The method of claim 89, wherein depletion is approached when the NTPs are at a concentration of no more than 5% of the concentration of NTPs that was present when the IVT reaction was initiated.

91. The method of claim 89, wherein depletion is approached when an NTP is at a concentration of no more than 5 mM.

92. The method of claim 91, wherein depletion is approached when an NTP is at a concentration of no more than 3 mM.

93. The method of claim 86, wherein the IVT reaction is supplemented continuously with NTPs during the course of manufacturing the RNA.

94. The method of claim 86-93, wherein each NTP is present at a concentration of 1-10 mM, 1-6 mM, 2-6 mM, or 3-6 mM when the IVT reaction is initiated.

95. The method of any one of claims 86-94, wherein the IVT reaction is supplemented with NTPs to maintain or restore the concentration at/to the concentration of NTPs that was present when the IVT reaction was initiated.

96. The method of any one of claims 86-94, wherein the IVT reaction is supplemented with NTPs to maintain the concentration of each NTP within a range of 20%-100%, 20%-75%, or 25%-50% that was present when the IVT reaction was initiated.

97. The method of any one of claims 86-94, wherein the total NTP concentration in the IVT reaction is maintained above a lower limit of 2 mM.

98. The method of claim 97, wherein the total NTP concentration in the IVT reaction is maintained at 10 mM to 20 mM.

99. A method of manufacturing an RNA using an in vitro transcription (IVT) reaction, said method comprising:
a) providing a DNA template comprising the nucleotide sequence of the RNA operationally linked to an RNA polymerase promoter;
b) adding the DNA template to a reaction vessel comprising an RNA polymerase and necessary reactants to initiate the IVT reaction; and
c) monitoring the IVT reaction by obtaining a series of spectra, wherein the spectra span a wavelength region suitable for monitoring the DNA template and/or the RNA polymerase during the IVT reaction.

100. The method of claim 99, wherein the spectra obtained in step (c) are used to monitor the amount of DNA template.

101. The method of claim 100, wherein the wavelength region obtained in step (c) comprises or consists of 500 cm⁻¹ to 710 cm⁻¹ to determine the amount of DNA template.

102. The method of any one of claims 99-101, wherein the spectra obtained in step (c) are used to monitor the amount of RNA polymerase, optionally wherein the RNA polymerase is SP6 RNA polymerase.

103. The method of claim 102, wherein the wavelength region obtained in step (c) comprises or consists of 780 cm⁻¹ to 1200 cm⁻¹ or 1430 cm⁻¹ to 1510 cm⁻¹ to determine the amount of RNA polymerase.

104. The method of any one of claims 99-103, wherein the method comprises adding a nuclease (e.g., DNase I) to terminate the IVT reaction.

105. The method of claim 104, wherein the spectra obtained in step (c) are used to monitor the amount or addition of the nuclease.

106. The method of claim 105, wherein the wavelength region obtained in step (c) comprises or consists of 450 cm⁻¹ to 520 cm⁻¹ to determine the amount or addition of the nuclease.

107. The method of any one of claims 99-106, wherein the method comprises adding a protease (e.g., proteinase K) to terminate the IVT reaction.

108. The method of claim 107, wherein the spectra obtained in step (c) are used to monitor the amount or addition of the protease.

109. The method of claim 108, wherein the wavelength region obtained in step (c) comprises or consists of 505 cm⁻¹ to 610 cm⁻¹, optionally 550 cm⁻¹ to 600 cm⁻¹, to determine the amount or addition of the protease.
